(19) United States
(12) Patent Application Publication
(10) Pub. No.: US 2004/0259118 A1
(43) Pub. Date: Dec. 23, 2004

(54) Methods and compositions for nucleic acid sequence analysis

(76) Inventor: Stephen C. Macevicz, Cupertino, CA (US)

Correspondence Address: STEPHEN C. MACEVICZ, 21890 RUCKER DRIVE, CUPERTINO, CA, 95014, US

(21) Appl. No.: 10/771,102

(22) Filed: Feb. 2, 2004

Related U.S. Application Data
(60) Provisional application No. 60/480,760, filed on Jun. 23, 2003.

(51) Int. Cl. C12Q 1/68; C12P 19/34

(52) U.S. Cl. 435/6; 435/91.2

(57) ABSTRACT

The invention provides methods, kits and materials for determinining simultaneously signature sequences of a population of tagged polynucleotides. Tags comprise at least two parts: a hybridization tag and a correlation tag. Size ladders of polynucleotide fragments are generated from the population of tagged polynucleotides that contain a plurality of size classes. After the size classes are separated, hybridization tags of the separated fragments are copied and labeled according to the identity of one or more bases at the ends of the fragments. In a preferred embodiment, the labeled tags are specifically hybridized to a plurality of random microarrays of tag complements. Signals generated at hybridization sites of different random microarrays are correlated by sequencing of the unique correlation tag. Signature sequences are determined by signals generated at hybridization sites having the same correlation tag on each of the plurality of random microarrays.

This application claims priority from U.S. provisional application Ser. No. 60/480,760 filed 23 Jun. 2003, which is incorporated herein by reference.

### FIELD OF THE INVENTION

The invention relates generally to compositions and methods for analyzing nucleic acids, and more particularly, to hybridization-based methods for characterizing nucleic acid populations.

### BACKGROUND

The availability of convenient and efficient methods for the accurate identification of genetic variation and expression patterns among large sets of genes is crucial for understanding the relationship between an organism's genetic make-up and the state of its health or disease, Collins et al, Science, 282: 682-689 (1998). In regard to expression analysis, several powerful techniques have been developed for such analyses that depend either on specific hybridization of probes to microarrays, e.g. Duggan et al, Nature Genetics, 21: 10-14 (1999); Hacia et al, Nature Genetics, 21: 42-47 (1999), or on the counting of tags or signatures of DNA fragments, e.g. Velculescu et al, Science, 270: 484-487 (1995); Brenner et al, Nature Biotechnology, 18: 630-634 (2000). While the former provides the advantages of scale and the capability of detecting a wide range of gene expression levels, such measurements are subject to variability relating to probe hybridization differences and cross-reactivity, element-to-element differences within microarrays, and microarray-to-microarray differences, Audic and Claverie, Genomic Res., 7: 986-995 (1997); Wittes et al, J. Natl. Cancer Inst. 91: 400-401 (1999); Brooks et al, American Pharmaceutical Review, 6: 102-105 (2003). On the other hand, the latter methods, which provide digital representations of abundance, are statistically more robust; they do not require repetition or standardization of counting experiments as counting statistics are well-modeled by the Poisson distribution, and the precision and accuracy of relative abundance measurements may be increased by increasing the size of the sample of tags or signatures counted. Unfortunately, however, this property is difficult to realize routinely because of the cost and complexity of implementing large scale efforts to analyze gene expression based on counting sequence tags.

In regard to assessing genetic variation, the primary technique for discovering and assessing sequence variation among individuals is massive and repetitive conventional sequencing, or so-called re-sequencing, e.g. Nickerson et al, Nature Genetics, 19: 233-240 (1998); Taillon-Miller and Kwok, Genome Res., 9: 499-505 (1999); Cargill et al, Nature Genetics, 22: 231-238 (1999). However, the cost of such projects can be prohibitive if any more than a very small fraction of a genome, such as a few “candidate” genes, is analyzed.

In the field of oncology, there is interest in measuring genome-wide copy number variation of local regions that characterize many cancers and that may have diagnostic or prognostic implications, e.g. Albertson et al, Nature Genetics, 34: 369-376 (2003). Presently, genome-wide scans of such variation are carried out using microarrays of BACs containing genomic DNA inserts, e.g. Snijders et al, Nature Genetics, 29: 263-264 (2001); Pinkel et al, Nature Genetics, 20: 207-211 (1998). These microarrays suffer from all the problems of conventional spotted microarrays used for gene expression analysis; thus, measurement of subtle variations in copy number is challenging.

In an attempt to improve the efficiency of large-scale sequencing efforts, Brenner, U.S. Pat. No. 5,763,175, describes methods of using oligonucleotide tags to transfer sequence information from templates to specific sites on an array of tag complements, or anti-tags. The method calls for attaching tags to sequencing templates, generating successively shortened amplification products of the templates with PCR primers that anneal to successively larger portions of the templates, copying and labeling the tags associated with each shortened amplification product, and then specifically hybridizing successively the amplified tags to an array of anti-tags to extract a signature sequence for each of the tagged templates. That is, the labeled tags serve as “proxies” for the templates in the hybridization reactions that provide the read-out of signature sequences. Such use of tags obviates the requirement for preparing and carrying out separate sequencing reactions for each template. The tags also permit mixtures of templates to be processed in one or a few reactions, since sequence information is extracted via the labeling and spatial separation of the tags on a hybridization array. Unfortunately, the processing steps disclosed in Brenner are difficult to carry out because they require either large numbers of different PCR primers and a large number of enzymatic steps and/or they require PCR amplifications with degenerate primers which often leads to the spurious amplification of mis-primed sequences. In an improvement to sequencing by proxy, Mao et al, International application WO 02/097113, proposed forming sets of different-sized fragments containing tags that would be separated into size classes. Each size class would be processed separately to generate collections of labeled tags that would be applied to a different spatially addressable microarray. Unfortunately, the use of separate spatially addressable microarrays either limits the number of sequences that can be simultaneously determined or increases the cost to prohibitive levels, and the disclosed schemes for generating separable size classes of fragments involve many steps that are technically challenging. Moreover, in all of the above tag-based schemes, “labeling by sampling” is used to provide populations of target polynucleotides wherein substantially every different polynucleotide has a different tag. This is accomplished by first forming a population of tag-polynucleotide conjugates between tags of a set that is vastly larger than the set of polynucleotides being labeled. A small sample of such conjugates are then taken to provide a population meeting the requirement that every different target polynucleotide have a different tag attached. Typically the set of tags is about a hundred times the size of the set of target polynucleotides; thus, a sample about 1% the size of the tag set will ensure that nearly every tag selected will be unique, and at the same time, ensure that nearly every target polynucleotide of the entire set of target polynucleotides will be selected. Unfortunately, while this leads to efficient and simultaneous labeling of large sets of polynucleotides, it also leads to very inefficient use of microarrays or other hybridization platforms that are used to obtain readouts by hybridizing copies of the tags from the sampled conjugates. This is because only a small percentage, e.g. 1%, of the hybridization sites of the microarrays or other platforms are used in the readout step.

In view of the above, it would be highly desirable if a signature sequencing technique were available for measuring gene expression, sequence variation, and genomic copy number variation that had the capability of massively parallel analysis of large numbers of templates or nucleic acid fragments, but that was free of the shortcomings of current techniques.

### SUMMARY OF THE INVENTION

Accordingly, objects of the invention include, but are not limited to, providing a method and compositions for analyzing gene expression; providing an improved method of labeling by sampling; providing a digital representation of relative abundances of polynucleotides in a complex population; providing a method for profiling gene expression of large numbers of genes simultaneously or identifying large numbers of polymorphic genes simultaneously; providing a method and compositions for re-sequencing predetermined or determinable regions of a genome in order to detect sequence variation; providing a method for generating sets of labeled oligonucleotide tags containing sequence information about a polynucleotide; providing a method for simultaneously generating signature sequences for a population of polynucleotides or sequencing templates; providing a method of identifying individual genomes by a set of signature sequences; providing a method of determining copy number variation within genomic DNA; and providing a method of determining associations between phenotypic traits and genotypes.

The invention accomplishes these and other objectives by providing compositions, kits, and methods that combine attachment of oligonucleotide tags to polynucleotides in a population by “labeling-by-sampling” and the use of distinguishable labels on the oligonucleotide tags attached to different classes of polynucleotide being monitored in a reaction. In one aspect, the invention provides a method of monitoring a population of polynucleotides in a reaction using oligonucleotide tags comprising the following steps: (i) forming tag-polynucleotide conjugates between polynucleotides of the population and oligonucleotide tags of a tag repertoire such that substantially every oligonucleotide tag of the repertoire forms a tag-polynucleotide conjugate with substantially every polynucleotide of the population; (ii) isolating a sample of the tag-polynucleotide conjugates such that not every different polynucleotide has a different oligonucleotide tag; (iii) conducting a reaction with a plurality of reaction outcomes on the sample, such that each tag-polynucleotide conjugate of the sample has a single reaction outcome; (iv) copying and labeling each oligonucleotide tag of a tag-polynucleotide conjugate according to its reaction outcome such that tag-polynucleotide conjugates having different reaction outcomes have oligonucleotide tags with distinguishable labels; (v) hybridizing the labeled oligonucleotide tags of each tag-polynucleotide conjugate with their respective complements under stringent hybridization conditions, the respective complements each being attached to a spatially discrete region on a solid phase support; and (vi) detecting signals from the labels of oligonucleotide tags hybridized to the solid phase support to determine reaction outcomes of the polynucleotides of the population. Preferably, in the step of isolating the sample size is in the range of from 5 percent to 250 percent of the size of the tag repertoire; and more preferably, in the range of from 10 percent to 200 percent, and still more preferably, in the range of from 25 percent to 150 percent.

In another aspect the invention provides a method of determining nucleotide sequences of a population of polynucleotides comprising the steps: (i) generating a size ladder of polynucleotide fragments by an extension reaction, each polynucleotide fragment of the same size ladder having an end and an oligonucleotide tag that is the same for every polynucleotide fragment of the size ladder, the oligonucleotide tag being selected from a minimally cross-hybridizing set of oligonucleotides; (ii) separating the polynucleotide fragments to form a plurality of fractions; (iii) copying and labeling the oligonucleotide tag of each polynucleotide fragment in each fraction according to the identity of one or more nucleotides at the end of such polynucleotide fragments; (iv) hybridizing the labeled oligonucleotide tags of each fraction with their respective complements under stringent hybridization conditions, the respective complements each being attached to a spatially discrete region on a solid phase support; and (v) detecting a sequence of signals from the labels of oligonucleotide tags hybridized to the solid phase support to determine the nucleotide sequences of the polynucleotides of the population. Preferably, in this aspect of the invention, oligonucleotide tags are attached to polynucleotides of the population by (a) forming tag-polynucleotide conjugates between polynucleotides of the population and oligonucleotide tags of a tag repertoire such that substantially every oligonucleotide tag of the repertoire forms a tag-polynucleotide conjugate with substantially every polynucleotide of the population; and (b) isolating a sample of the tag-polynucleotide conjugates such that not every different polynucleotide has a different oligonucleotide tag.

In another aspect, the invention provides a method of labeling polynucleotides in a population by the steps of (i) forming tag-polynucleotide conjugates between polynucleotides of the population and oligonucleotide tags of a tag repertoire such that substantially every oligonucleotide tag of the repertoire forms a tag-polynucleotide conjugate with substantially every polynucleotide of the population; and (ii) isolating a sample of the tag-polynucleotide conjugates such that not every different polynucleotide has a different oligonucleotide tag. Again, preferably, in the step of isolating the sample size is in the range of from 5 percent to 250 percent of the size of the tag repertoire; and more preferably, in the range of from 10 percent to 200 percent, and still more preferably, in the range of from 25 percent to 150 percent.

In yet another aspect, the invention provides a method of measuring relative genomic amplification over a genome comprising the following steps: (i) providing a partition of a genome, the partition comprising a plurality of fragments uniformly distributed over the genome, each fragment having a genomic location; (ii) generating a signature sequence from each fragment; (iii) tabulating signature sequences of the fragments at each genomic location; and (iv) determining relative genomic amplification by a relative abundance of each fragment from the tabulated signature sequences.

In another aspect, the invention provides a method of determining single nucleotide polymorphisms uniformly distributed over a genome, the method comprising the steps of: (i) providing a partition of a genome, the partition comprising a plurality of fragments uniformly distributed over the genome, each fragment having a genomic location; (ii) generating a signature sequence from each fragment; (iii) tabulating signature sequences of the fragments at each genomic location; and (iv) determining the set of single nucleotide polymorphisms from the tabulated signature sequences. In a related aspect, the invention further provides method of determining frequencies of single nucleotide polymorphisms uniformly distributed over a plurality genomes, the method comprising the steps of: (i) providing a partition of a plurality of genomes, the partition comprising a plurality of fragments uniformly distributed over the genomes, each fragment having a genomic location; (ii) generating a signature sequence from each fragment; (iii) tabulating signature sequences of the fragments at each genomic location; and (iv) determining frequencies of single nucleotide polymorphisms from the tabulated signature sequences.

### BRIEF DESCRIPTION OF THE DRAWINGS

FIGS. 1A-1F illustrate one embodiment of the present invention.

FIGS. 2A-2B illustrate the steps of generating a library of tag-polynucleotide conjugates.

FIG. 3 illustrates an apparatus for hybridizing labeled tags to an array of microbeads.

FIG. 4 illustrate the application of the invention to genome-wide genotyping.

### DEFINITIONS

As used herein, “addressable” or “addressed” in reference to tag complements means that the nucleotide sequence, or perhaps other physical or chemical characteristics, of a tag complement can be determined from its address, i.e. a one-to-one correspondence between the sequence or other property of the tag complement and a spatial location on, or characteristic of, the solid phase support to which it is attached. Preferably, an address of a tag complement is a spatial location, e.g. the planar coordinates of a particular region containing copies of the tag complement. However, tag complements may be addressed in other ways too, e.g. by microparticle size, shape, color, frequency of micro-transponder, or the like, e.g. Chandler et al, PCT publication WO 97/14028.

As used herein, “allele frequency” in reference to a genetic locus, a sequence marker, or the site of a nucleotide means the frequency of occurrence of a sequence or nucleotide at such genetic loci or the frequency of occurrence of such sequence marker, with respect to a population of individuals. In some contexts, an allele frequency may also refer to the frequency of sequences not identical to, or exactly complementary to, a reference sequence.

As used herein, “amplicon” means the product of an amplification reaction. That is, it is a population of polynucleotides, usually double stranded, that are replicated from one or more starting sequences. The one or more starting sequences may be one or more copies of the same sequence, or it may be a mixture of different sequences. Preferably, amplicons are produced either in a polymerase chain reaction (PCR) or by replication in a cloning vector.

“Chromatography” or “chromatographic separation” as used herein means or refers to a method of analysis in which the flow of a mobile phase, usually a liquid, containing a mixture of compounds, e.g. molecular tags, promotes the separation of such compounds based on one or more physical or chemical properties by a differential distribution between the mobile phase and a stationary phase, usually a solid. The one or more physical characteristics that form the basis for chromatographic separation of analytes, such as molecular tags, include but are not limited to molecular weight, shape, solubility, pKa, hydrophobicity, charge, polarity, and the like. In one aspect, as used herein, “high pressure (or performance) liquid chromatography” (“HPLC”) refers to a liquid phase chromatographic separation that (i) employs a rigid cylindrical separation column having a length of up to 300 mm and an inside diameter of up to 5 mm, (ii) has a solid phase comprising rigid spherical particles (e.g. silica, alumina, or the like) having the same diameter of up to 5 μm packed into the separation column, (iii) takes place at a temperature in the range of from 35° C. to 80° C. and at column pressure up to 150 bars, and (iv) employs a flow rate in the range of from 1 μL/min to 4 mL/min. Preferably, solid phase particles for use in HPLC are further characterized in (i) having a narrow size distribution about the mean particle diameter, with substantially all particle diameters being within 10% of the mean, (ii) having the same pore size in the range of from 70 to 300 angstroms, (iii) having a surface area in the range of from 50 to 250 m²/g, and (iv) having a bonding phase density (i.e. the number of retention ligands per unit area) in the range of from 1 to 5 per nm². Exemplary reversed phase chromatography media for separating molecular tags include particles, e.g. silica or alumina, having bonded to their surfaces retention ligands, such as phenyl groups, cyano groups, or aliphatic groups selected from the group including C₈ through C₁₈. Chromatography in reference to the invention includes “capillary electrochromatography” (“CEC”), and related techniques. CEC is a liquid phase chromatographic technique in which fluid is driven by electroosmotic flow through a capillary-sized column, e.g. with inside diameters in the range of from 30 to 100 μm. CEC is disclosed in Svec, Adv. Biochem. Eng. Biotechnol. 76: 1-47 (2002); Vanhoenacker et al, Electrophoresis, 22: 4064-4103 (2001); and like references. CEC column may use the same solid phase materials as used in conventional reverse phase HPLC and additionally may use so-called “monolithic” non-particular packings. In some forms of CEC, pressure as well as electroosmosis drives an analyte-containing solvent through a column.

“Complement” or “tag complement” as used herein in reference to oligonucleotide tags refers to an oligonucleotide to which an oligonucleotide tag specifically hybridizes to form a perfectly matched duplex or triplex. In embodiments where specific hybridization results in a triplex, the oligonucleotide tag may be selected to be either double stranded or single stranded. Thus, where triplexes are formed, the term “complement” is meant to encompass either a double stranded complement of a single stranded oligonucleotide tag or a single stranded complement of a double stranded oligonucleotide tag.

“Kit” as used herein refers to any delivery system for delivering materials. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“Labeling by sampling” means a process of (i) forming tag-polynucleotide conjugates between polynucleotides of the population and oligonucleotide tags of a tag repertoire such that substantially every oligonucleotide tag of the repertoire forms a tag-polynucleotide conjugate with substantially every polynucleotide of the population; and (ii) isolating a sample of the tag-polynucleotide conjugates such that not every different polynucleotide has a different oligonucleotide tag. Preferably, in the step of isolating the sample size is in the range of from 5 percent to 250 percent of the size of the tag repertoire; and more preferably, in the range of from 10 percent to 200 percent, and still more preferably, in the range of from 25 percent to 150 percent.

“Nucleobase” means a nitrogen-containing heterocyclic moiety capable of forming Watson-Crick type hydrogen bonds with a complementary nucleobase or nucleobase analog, e.g. a purine, a 7-deazapurine, or a pyrimidine. Typical nucleobases are the naturally occurring nucleobases adenine, guanine, cytosine, uracil, thymine, and analogs of naturally occurring nucleobases, e.g. 7-deazaadenine, 7-deaza azaadenine, 7-deazaguanine, 7-deaza azaguanine, inosine, nebularine, nitropyrrole, nitroindole, 2-amino-purine, 2,6-diaminopurine, hypoxanthine, pseudouridine, pseudocytidine, pseudoisocytidine, 5-propynylcytidine, isocytidine, isoguanine, 2-thiopyrimidine, 6-thioguanine, 4-thiothymine, 4-thiouracil, O6-methylguanine, N6-methyl-adenine, O4-methylthymine, 5,6-dihydrothymine, 5,6-dibydrouracil, 4-methylindole, and ethenoadenine, e.g. Fasman, Practical Handbook of Biochemistry and Molecular Biology, pp. 385-394, CRC Press, Boca Raton, Fla. (1989).

“Nucleoside” means a compound comprising a nucleobase linked to a C-1′ carbon of a ribose sugar or analog thereof. The ribose or analog may be substituted or unsubstituted. Substituted ribose sugars include, but are not limited to, those riboses in which one or more of the carbon atoms, preferably the 3′-carbon atom, is substituted with one or more of the same or different substituents such as —R, —OR, —NRR or halogen (e.g., fluoro, chloro, bromo, or iodo), where each R group is independently —H, C1-C6 alkyl or C3-C14 aryl. Particularly preferred riboses are ribose, 2′-deoxyribose, 2′,3′-dideoxyribose, Y-haloribose (such as 3′-fluororibose or 3′-chlororibose) and 3′-alkylribose. Typically, when the nucleobase is A or G, the ribose sugar is attached to the N9-position of the nucleobase. When the nucleobase is C, T or U, the pentose sugar is attached to the N′-position of the nucleobase (Komberg and Baker, DNA Replication, 2 d Ed., Freeman, San Francisco, Calif., (1992)). Examples of ribose analogs include arabinose, 2′-O-methyl ribose, and locked nucleoside analogs (e.g., WO 99/14226), for example, although many other analogs are also known in the art.

“Nucleotide” means a phosphate ester of a nucleoside, either as an independent monomer or as a subunit within a polynucleotide. Nucleotide triphosphates are sometimes denoted as “NTP”, “dNTP” (2′-deoxypentose) or “ddNTP” (2′,3′-dideoxypentose) to particularly point out the structural features of the ribose sugar. “Nucleoside 5′-triphosphate” refers to a nucleotide with a triphosphate ester group at the 5′ position. The triphosphate ester group may include sulfur substitutions for one or more phosphate oxygen atoms, e.g. α-thionucleoside 5′-triphosphates.

“Oligonucleotide” as used herein means linear oligomers of natural or modified nucleosidic monomers linked by phosphodiester bonds or analogs thereof. Oligonucleotides include deoxyribonucleosides, ribonucleosides, anomeric forms thereof, peptide nucleic acids (PNAs), and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units, e.g. 40-60. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides of the invention comprise the four natural deoxynucleotides; however, they may also comprise ribonucleosides or non-natural nucleotide analogs. It is clear to those skilled in the art when oligonucleotides having natural or non-natural nucleotides may be employed in the invention. For example, where processing by an enzyme is called for, usually oligonucleotides consisting of natural nucleotides are required. Likewise, where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, N.Y., 1989), and like references.

“Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex. Conversely, a “mismatch” in a duplex between a tag and an oligonucleotide means that a pair or triplet of nucleotides in the duplex or triplex fails to undergo Watson-Crick and/or Hoogsteen and/or reverse Hoogsteen bonding. As used herein, “stable duplex” between complementary oligonucleotides or polynucleotides means that a significant fraction of such compounds are in duplex or double stranded form with one another as opposed to single stranded form. Preferably, such significant fraction is at least ten percent of the strand in lower concentration, and more preferably, thirty percent.

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex. Conversely, a “mismatch” in a duplex between a tag and an oligonucleotide means that a pair or triplet of nucleotides in the duplex or triplex fails to undergo Watson-Crick and/or Hoogsteen and/or reverse Hoogsteen bonding.

“Relative genomic amplification” means a condition wherein local portions of a genome are present in higher or lower copy number than that observed in a normal cell. In one aspect, this means any deviation from a normal diploid complement of chromosomal DNA.

The term “sample” in the present specification and claims is used in a broad sense. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

As used herein “sequence determination” or “determining a nucleotide sequence” in reference to polynucleotides includes determination of partial as well as full sequence information of the polynucleotide. That is, the term includes sequence comparisons, fingerprinting, and like levels of information about a target polynucleotide, as well as the express identification and ordering of nucleosides, usually each nucleoside, in a target polynucleotide. The term also includes the determination of the identity, ordering, and locations of one, two, or three of the four types of nucleotides within a target polynucleotide. For example, in some embodiments sequence determination may be effected by identifying the ordering and locations of a single type of nucleotide, e.g. cytosines, within the target polynucleotide “CATCGC . . . ” so that its sequence is represented as a binary code, e.g. “100101 . . . ” for “C-(not C)-(not C)—C-(not C)—C . . . ” and the like.

As used herein “signature sequence” means a sequence of nucleotides derived from a polynucleotide such that the ordering of nucleotides in the signature is the same as their ordering in the polynucleotide and the sequence contains sufficient information to identify the polynucleotide in a population. Signature sequences may consist of a segment of consecutive nucleotides (such as, (a,c,g,t,c) of the polynucleotide “acgtcggaaatc”), or it may consist of a sequence of every second nucleotide (such as, (c,t,g,a,a,) of the polynucleotide “acgtcggaaatc”), or it may consist of a sequence of nucleotide changes (such as, (a,c,g,t,c,g,a,t,c) of the polynucleotide “acgtcggaaatc”), or like sequences.

As used herein, the term “complexity” in reference to a population of polynucleotides means the number of different species of polynucleotide present in the population.

As used herein, “ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically.

As used herein, “microarray” refers to a solid phase support having a planar surface, which carries an array of nucleic acids, each member of the array comprising identical copies of an oligonucleotide or polynucleotide immobilized to a spatially defined region or site, which does not overlap with those of other members of the array; that is, the regions or sites are spatially discrete. Spatially defined hybridization sites may additionally be “addressable” in that its location and the identity of its immobilized oligonucleotide are known or predetermined, for example, prior to its use. Typically, the oligonucleotides or polynucleotides are single stranded and are covalently attached to the solid phase support. The density of non-overlapping regions containing nucleic acids in a microarray is typically greater than 100 per cm², and more preferably, greater than 1000 per cm². Microarray technology is reviewed in the following references: Schena et al, Trends in Biotechnology, 16: 301-306 (1998); Southern, Current Opin. Chem. Biol., 2: 404-410 (1998); Nature Genetics Supplement, 21: 1-60 (1999). As used herein, “random microarray” refers to a microarray whose spatially discrete regions of oligonucleotides or polynucleotides are not spatially addressed. That is, the identity of the attached oligonucleoties or polynucleotides is not discernable, at least initially, from its location. Preferably, random microarrays are planar arrays of microbeads wherein each microbead has attached a single kind of hybridization tag complement. Arrays of microbeads may be formed in a variety of ways, e.g. Brenner et al, Nature Biotechnology; 18: 630-634 (2000); Tulley et al, U.S. Pat. No. 6,133,043; Stuelpnagel et al, U.S. Pat. No. 6,396,995; Chee et al, U.S. Pat. No. 6,544,732; and the like. An important advantage of random microarrays of bead is that combinatorial tags may be synthesized on the beads at very low cost using conventional “split and mix” strategies.

As used herein, “genetic locus,” or “locus” in reference to a genome or target polynucleotide, means a contiguous subregion or segment of the genome or target polynucleotide. As used herein, genetic locus, or locus, may refer to the position of a gene or portion of a gene in a genome, or it may refer to any contiguous portion of genomic sequence whether or not it is within, or associated with, a gene. Preferably, a genetic locus refers to any portion of genomic sequence from a few tens of nucleotides, e.g. 10-30, in length to a few hundred nucleotides, e.g. 100-300, in length.

As used herein, “sequence marker” means a portion of nucleotide sequence at a genetic locus. A sequence marker may or may not contain one or more single nucleotide polymorphisms, or other types of sequence variation, relative to a reference or control sequence. In accordance with the invention, a sequence marker may be interrogated by specific hybridization of an isostringency probe.

“Specific” or “specificity” in reference to the binding of one molecule to another molecule, such as a probe for a target polynucleotide, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak noncovalent chemical interactions, such as Van der Waal forces, hydrogen bonding, ionic and hydrophobic interactions, and the like, dominate the interaction of the molecules. As used herein, “stable complex” in reference to two or more molecules means that such molecules form noncovalently linked aggregates, e.g. by specific binding, that under assay conditions are thermodynamically more favorable than a non-aggregated state.

“Spectrally resolvable” in reference to a plurality of fluorescent labels means that the fluorescent emission bands of the labels are sufficiently distinct, i.e. sufficiently non-overlapping, that molecular tags to which the respective labels are attached can be distinguished on the basis of the fluorescent signal generated by the respective labels by standard photodetection systems, e.g. employing a system of band pass filters and photomultiplier tubes, or the like, as exemplified by the systems described in U.S. Pat. Nos. 4,230,558; 4,811,218, or the like, or in Wheeless et al, pgs. 21-76, in Flow Cytometry: Instrumentation and Data Analysis (Academic Press, New York, 1985).

As used herein, the term “Tm” is used in reference to the “melting temperature.” The melting temperature is the temperature at which a population of double-stranded nucleic acid molecules becomes half dissociated into single strands. Several equations for calculating the Tm of nucleic acids are well known in the art. As indicated by standard references, a simple estimate of the T,, value may be calculated by the equation. Tm=81.5+0.41 (% G+C), when a nucleic acid is in aqueous solution at I M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tm.

“Terminator,” or “chain terminator,” means a nucleotide that can be incorporated into a primer by a polymerase extension reaction, wherein the nucleotide prevents subsequent incorporation of nucleotides to the primer and thereby halts polymerase-mediated extension. Typical terminators lack a 3′-hydroxyl substituent and include 2′,3′-dideoxyribose, 2′,3′-didebydroribose, and 2′,3′-dideoxy-3′-baloribose, e.g. 3′-deoxy-3′-fluoro-ribose or 2′,3′-dideoxy-3′-fluororibose, for example. Alternatively, a ribofuranose analog can be used, such as 2′,3′-dideoxy-β-D-ribofuranosyl, β-D-arabinofuranosyl, 3′-deoxy-β-D-arabinofuranosyl, 3′-arnino-2′,3′-dideoxy-β-D-ribofaranosyl, and 2,3′-dideoxy-3′-fluoro-β-D-ribofuranosyl. A variety of terminators are disclosed in the following references: Chidgeavadze et al., Nucleic Acids Res., 12: 1671-1686 (1984); Chidgeavadze et al., FEBS Lett., 183: 275-278 (1985); Izuta et al, Nucleosides & Nucleotides, 15: 683-692 (1996); and Krayevsky et al, Nucleosides & Nucleotides, 7: 613-617 (1988). Nucleotide terminators also include reversible nucleotide terminators, e.g. Metzker et al. Nucleic Acids Res., 22(20):4259 (1994). Terminators of particular interest are terminators having a capture moiety, such as biotin, or a derivative thereof, e.g. Ju, U.S. Pat. No. 5,876,936, which is incorporated herein by reference. As used herein, a “predetermined terminator” is a terminator that basepairs with a pre-selected nucleotide of a template.

As used herein, “uniform” in reference to spacing or distribution means that a spacing between objects, such as sequence markers, or events may be approximated by an exponential random variable, e.g. Ross, Introduction to Probability Models, 7^{th} edition (Academic Press, New York, 2000). In regard to spacing of sequence markers in a mammalian genome, it is understood that there are significant regions of repetitive sequence DNA in which a random sequence model of the genomic DNA does not hold. “Uniform” in reference to spacing of sequence markers preferably refers to spacing in uniques sequence regions, i.e. non-repetitive sequence regions, of a genome.

### DETAILED DESCRIPTION OF THE INVENTION

The invention provides a method of labeling by sampling that includes the use of different labels on oligonucleotide tags that permit the detection of “doubles,” that is, tag-polynucleotide conjugates wherein the same tag is attached to two or more different polynucleotides. This situation occurs more frequently the greater a sample size. In particular, Brenner et al (citations above) teach that substantially every polynucleotide of a sample will have a unique tag provided that the size of the sample is small, e.g. 1%, of the size of the tag repertoire used. The present invention permits far larger samples to be taken as long as the tags for different classes of polynucleotide (for example, those ending in “A,” those ending in “C,” etc.) have distinguishable labels in a readout step. In a sequence of measurements, where doubles exist, eventually two or more tags will be produced with different labels that will hybridize to the same hybridization site. This ambiguous signal indicates a double, and signals from such sites are then disregarded. The advantage of the invention is that when an addressable array is used as a readout device, a much large fraction of its sites are used, e.g. 0.65-0.70 for a 100% sample, versus 0.01 for a 1% sample.

In one aspect, the invention provides a method of simultaneously sequencing polynucleotides in a complex mixture by using oligonucleotide tags to shuttle sequence information obtained from the polynucleotides to discrete hybridization sites on one or more solid phase supports, such as a plurality of random microarrays. In a single reaction tube, a population of template sequences (or equivalently, target polynucleotides) are subjected to a reaction or a series of reactions that produces a mixture of labeled oligonucleotide tags such that each tag is derived from (and therefore is associated with) a different template (or target polynucleotide). The labels on the oligonucleotide tags identifies or provides information about one or more nucleotides of the template sequence with which it is associated. For example, in one embodiment, labels may each be one of four fluorescent dyes, each with a different emission band, so that there is a one-to-one correspondence between a fluorescent dye and whether a nucleotide at a given position on a template is A, C, G, or T. In accordance with the method, usually, a separate reaction or series of reactions is implemented for identifying nucleotides at different positions on template sequences.

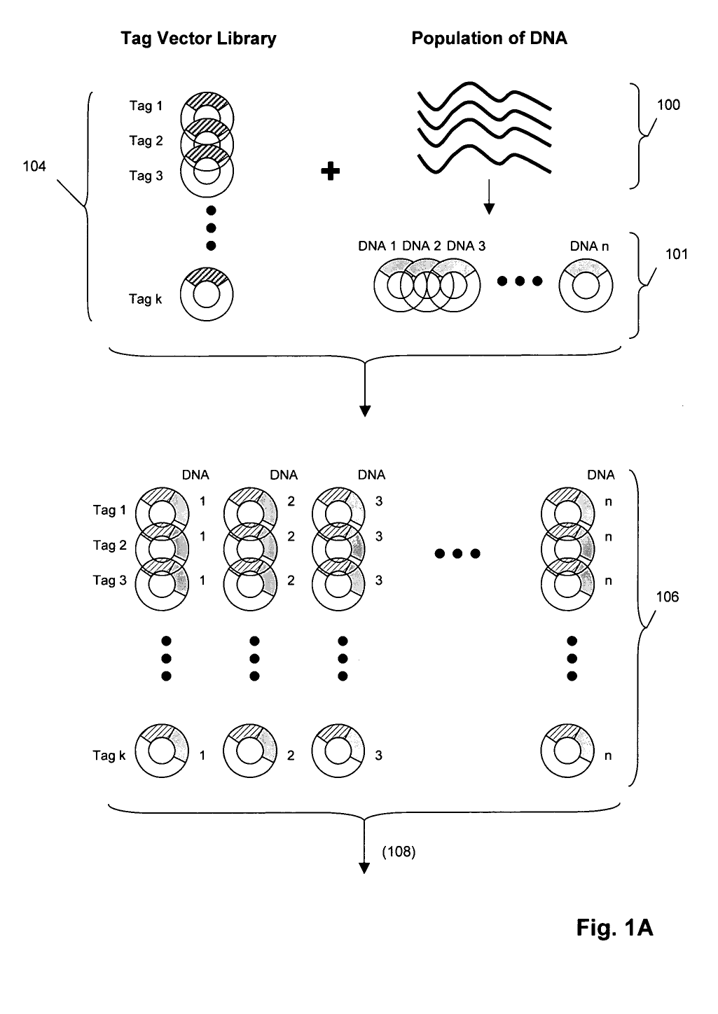

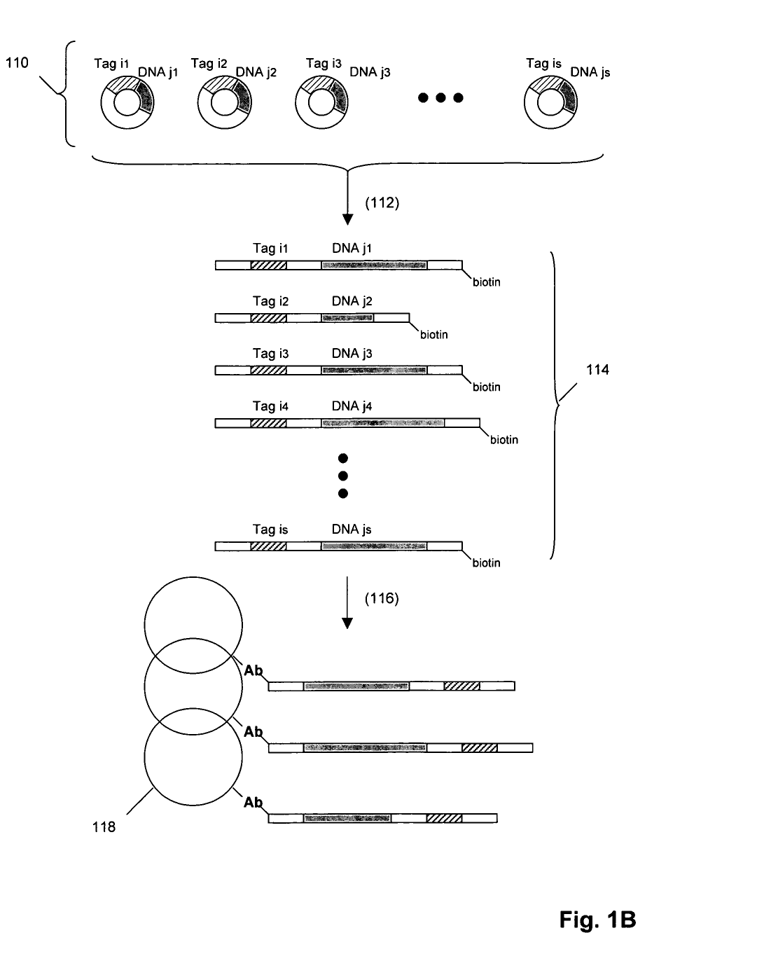

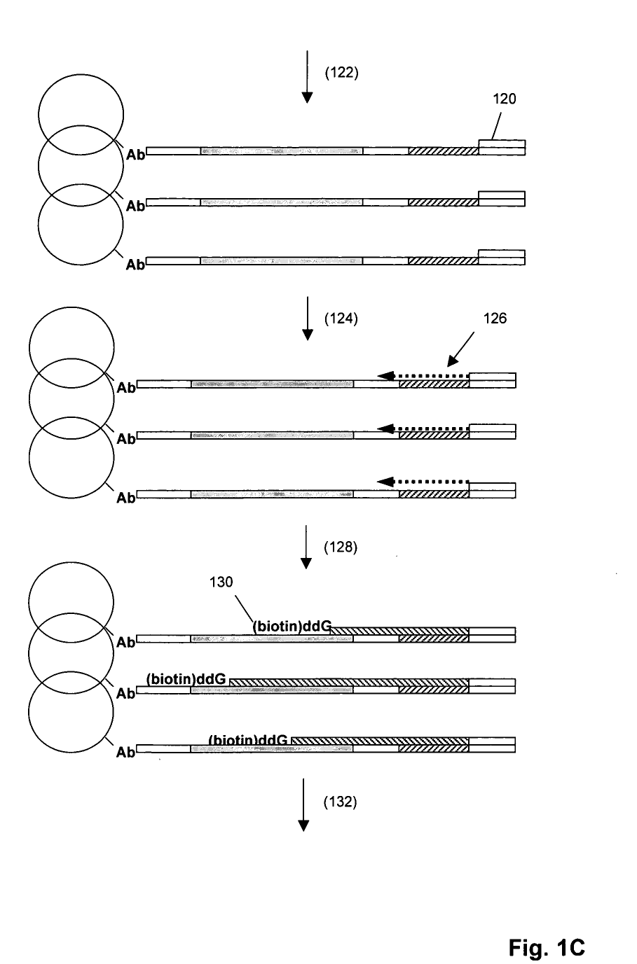

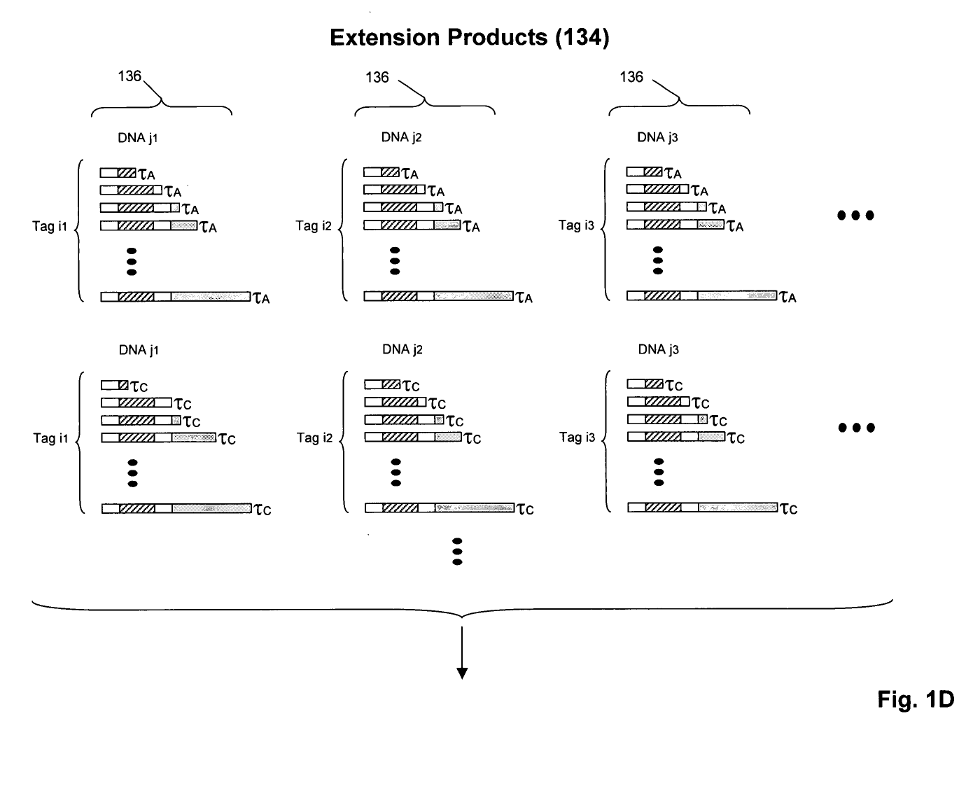

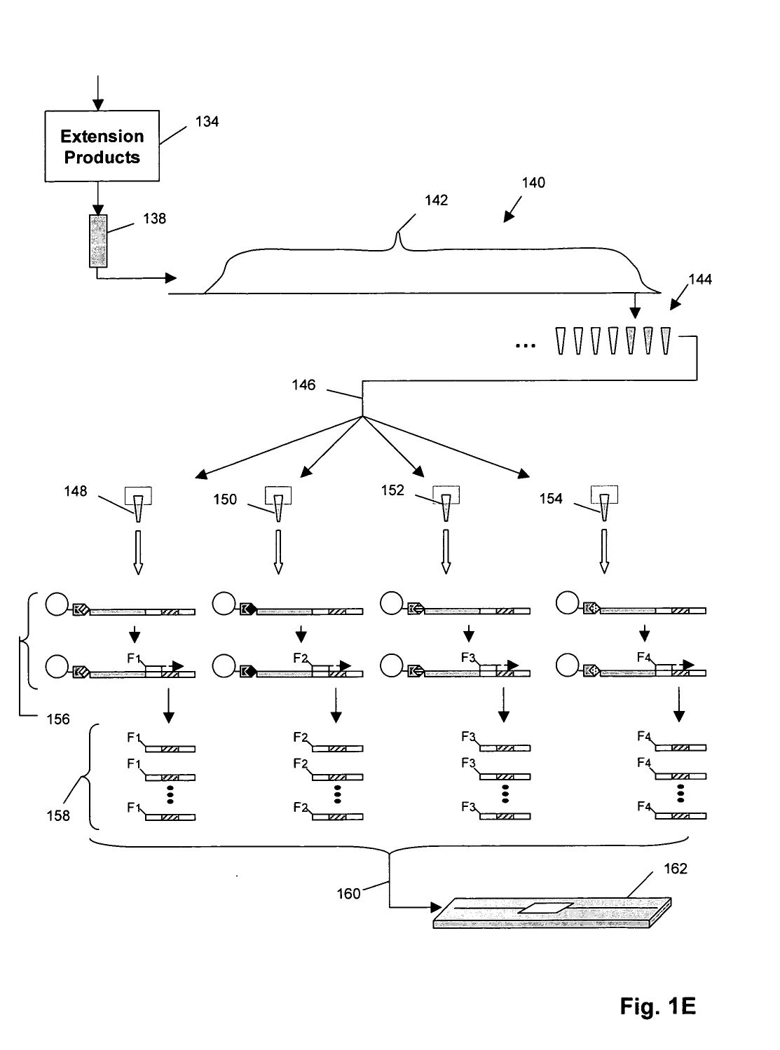

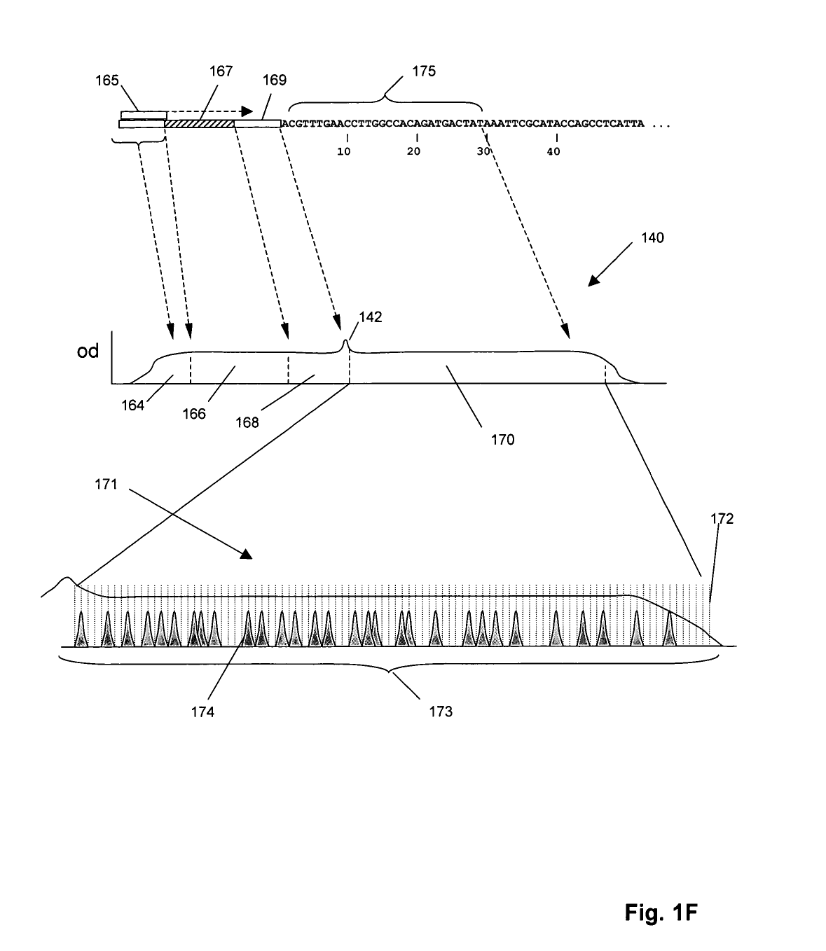

One aspect of the invention is illustrated in FIGS. 1A-1F. Polynucleotides of a complex mixture (100) are conjugated (102) to oligonucleotide tags of a repertoire of tags (104) to form a population of tag-polynucleotide conjugates (106), as described in Brenner et al, U.S. Pat. No. 5,846,719, and Brenner et al, Proc. Natl. Acad. Sci., 97: 1665-1670 (2000), which are incorporated by reference. (For example, the DNA is excised from vectors (101) and inserted into the vectors containing tag repertoire (104) using conventional molecular biology techniques, e.g. Sambrook et al, Molecular Cloning: A Laboratory Manual, 2^{nd} Edition (Cold Spring Harbor Laboratory)). In accordance with those references, by selecting a repertoire of tags having a substantially larger number of distinct species than the size of the population of polynucleotides, a sample of conjugates can be selected which is large enough so that all of the different species of polynucleotide are included, but which is also small enough so that the overwhelming majority of the polynucleotides will each have a unique tag. A typical sample size to achieve this result is about one percent of the total number of different kinds of tags in the repertoire of tags employed. An important aspect of the present invention is based on the observation that when oligonucleotide tags representing different events, e.g. different nucleotides at the same locus of a template, have distinguishable labels, then the occurrence of so-called “doubles” (i.e., two different polynucleotides having the same oligonucleotide tag) can be detected by the presence of two distinct labels at the same hybridization site. Thus, the sample size may be much larger than that taught in the above references because “doubles” can simply be discarded or ignored during a detection step. The following example illustrates how this increases sequencing efficiency. If a repertoire of tags consisted of 100,000 oligonucleotide tags and detection was carried out on a 100,000-element microarray, one percent sampling means that only 1000 of the microarray elements are used in any given experiment. However, if elements that simultaneously accept differently labeled oligonucleotide tags can be detected, then (for example) a one hundred percent sample gives about 60% uniquely labeled polynucleotides and about 40% doubles. The 40% doubles can be discarded or ignored; the 60% uniquely tagged polynucleotides generate unambiguous signals for signature sequences. 60,000 of the microarray elements are used, rather than only 1000.

Returning to FIG. 1A, a sample (110, FIG. 1B) is taken (108) form the population of tag-polynucleotide conjugates (106). Vectors containing tags (104) are engineered to have flanking primer binding sites so that tag-polynucleotide conjugates from sample (110) can be conveniently replicated and modified, e.g. by using biotinylated primers, as shown. Tag-polynucleotide conjugates of sample (110) are replicated so that a biotin, or other capture moiety, is attached to one end of the replicated sequences (114). The sequences (114) are then captured by a capture agent, such as avidin or streptavidin, attached to solid phase support (118), such as streptavidinated magnetic beads, e.g. Dynal. Sequences (114) are washed, after which primers (120) are annealed (122) to the primer binding site distal to solid phase support (118). Primers (120) are then extended (124) with a conventional DNA polymerase (126) in the presence of one or more terminators (130) using the captured fragment as a template so that size ladders of terminated fragments are generated. As used herein, the term “template-dependent extension” refers to a method of extending a primer on a template nucleic acid that produces an extension product that is complementary to the template nucleic acid. Preferably, extension reaction conditions are selected, e.g. by routine experimentation, to produce fragments having lengths ranging from the size of primers (120) to 50-100 nucleotides. Preferably, four different terminators are employed so that fragments are produced in the same reaction terminating with terminators for each of the four natural nucleotides. In FIG. 1C, only the terminator dideoxyguanosine (130) having a biotin attached is shown. In further preference, different terminators have different capture moieties attached so that samples of each of the four sets of terminated fragments can be removed separately from the extension reaction mixture. Many different terminator-capture moiety combinations are available. Preferably, dideoxynucleoside triphosphates are used as terminators. In one aspect, capture moieties may be attached to such terminators derivatized with an alkynylamino group, as taught by Hobbs et al, U.S. Pat. No. 5,047,519 and Taing et al, International patent publication WO 02/30944, which are incorporated herein by reference. Preferable capture moieties include biotin or biotin derivatives, such as desbiotin, which are captured with streptavidin or avidin or commercially available antibodies, and dinitrophenol, digoxigenin, fluorescein, and rhodamine, all of which are available as NHS-esters that may be reacted with alkynylamino-derivatized terminators. These reagents as well as antibody capture agents for these compounds are available for Molecular Probes, Inc. (Eugene, Oreg.). It is noted that prior to using terminators having biotin attached, if solid phase support (118) is avidinated or streptavidinated, it may be saturated with free biotin to prevent the terminator from binding to available sites on the avidinated or streptavidinated support. A preferred composition of the invention is a mixture of terminators with different capture moieties for use in the extension reaction. More preferably, this composition comprises the four dideoxynucleoside triphosphates (ddATP, ddCTP, ddGTP, and ddTTP) each having a different capture moiety attached selected from the group consisting of biotin, desbiotin, dinitrophenol, digoxigenin, fluorescein, and rhodamine. Kits of the invention include this mixture of terminators together with their respective capture agent attached to a solid phase support, such as magnetic beads.

After the extension reaction is completed, the extension products may be washed and then melted (132) from solid phase support (118). As illustrated in FIG. 1D, extension products (134) include size ladders (136) for every tag-polynucleotide conjugate of sample (110). Each size ladder (136) has four subsets, one for each set of fragments ending with terminator for A (“τ_{A}”), C (“τ_{C}”), G (“τ_{G}”), and T (“τ_{T}”). After isolation, extension products (134) are separated by size using a conventional preparative separation technique, such as chromatography or gel electrophoresis. Preferably, extension products (134) are separated by denaturing HPLC (dHPLC)(138), for example, using a column and instrument such as DNASep and Wave™ system (Transgenomic, Omaha, Nebr.). Guidance for selecting an appropriate column, instrument, and condition for separation is found in the following references that are incorporated by reference: Haefele et al, Application Note 103 (2000, Transgenomic, Omaha, Nebr.); Premstaller et al, PharmaGenomics, 20-37 (February, 2003); Xiao et al, Human Mutation, 17: 439474 (2001); Warren et al, Molecular Biotechnology, 4: 179-199 (1995); Huber et al, Anal. Chem. 67: 578-585 (1995); Dickman et al, Anal. Biochem., 284: 164-167 (2000); Oefiner et al, Anal. Biochem., 223: 3946 (1994).

Because of the large heterogeneous population of fragments the separation produces a continuouse separation profile in which individual peaks corresponding to individual size classes are not identifiable by a measurement such as optical density, or the like, that measure total polynucleotide. However, as illustrated in FIG. 1F, there is a correlation between fragment size and position in separation profile (140). Generally, region (164) corresponds to flanking primer (165), region (166) corresponds to fragments terminated in tag sequence (167), region (168) corresponds to fragments terminated in internal primer binding site (169), and region (170) corresponds to fragments terminated in signature sequence region (175). A size marker oligonucleotide may be added to the extension products to mark the boundary between internal primer binding site (169) and signature sequence region (175). Such a marker is detected as optical density peak (142) in the separation profile. In particular, with in the bulk of fragments, those peaks (174) from a single size ladder (173) are separated. It is desirable to carry out as few hybridizations as possible to identify nucleotide sequences; thus, fractions are preferably collected only from portion (170) of separation profile (140).

Returning to FIG. 1E, fractions (144) of the separated fragments are collected. Preferably, the amount of eluent collected in each fraction is selected so that the portion of the separation profile containing the signature sequence, i.e. region (170), corresponds to a total number of fractions in the range of from about 30 to 200. Each fraction is treated (146) with the four different capture agents to isolated fragments having different terminators (148, 150, 152, and 154, respectively), after which labeled primers are annea (156) to the captured fragments and are extended in a cycled extension reaction to generate labeled tags (158). Preferably, labels F₁, F₂, F₃, and F₄ are spectrally resolvable fluorescent dyes. The labeled tags are then hybridized (160) to array (162) and detected.

Preferably, the number of fractions is sufficiently large so that for a given size ladder no more than one peak will span, or be contained in, a fraction corresponding to a particular migration time. Under these conditions, a signature sequence is determined at each hybridization site, e.g. a single microbead, by observing a sequence of signals, e.g. from different fluorescent dyes, generated at the site by successive hybridizations of labeled hybridization tags.

A feature of the invention is the generation of a size ladder of polynucleotide fragments for each tag-polynucleotide conjugate of the sample. As used herein, the term “size ladder” in reference to a tag-polynucleotide conjugate means a series of polynucleotide fragments generated from the tag-polynucleotide conjugate, wherein each polynucleotide fragment of the same size ladder has the same oligonucleotide tag attached and wherein the lengths of each of the polynucleotide fragments within a size ladder differ from one another by a predetermined number of nucleotides. That is, the a size ladder may be generated by removing predetermined numbers of nucleotides from a tag-polynucleotide conjugate, or it may be generated by extending a primer a predetermined number of nucleotides on a template derived from a tag-polynucleotide conjugate. For example, in a simple case, a size ladder is generated by successively removing a single nucleotide from the end of the polynucleotide of a tag-polynucleotide conjugate, so that the size ladder consists of a series of polynucleotide fragments each differing in length from its closest neighbor by one nucleotide. However, it is not necessary that the size classes of a size ladder differ in length by multiples of a constant number of nucleotides. A size ladder may consist of any series of polynucleotide fragments whose ends terminate at any of a collection of nucleotide positions that are the same for all the different tag-polynucleotide conjugates of a mixture. The important feature is that the differences in fragment sizes within a size ladder not vary from fragment to fragment so that a correspondence exists between the signature sequence generated and the polynucleotide it is derived from. Preferably, the size differences between fragments of a size ladder are predetermined and are the same for all the tag-polynucleotide conjugates. More preferably, the fragments of a size ladder each differ in length by one nucleotide, and preferably, such fragments are generated by extending a primer by a nucleic acid polymerase in the presence of one or more terminators that have a capture moiety attached. Such extension are carried out using conventional sequencing reactions, e.g. Sambrook et al, Molecular Cloning: A Laboratory Manual, Second Edition (Cold Spring Harbor Laboratory Press, 1989).

In accordance with the invention, generation of size ladders for every tag-polynucleotide conjugate of a sample produces a mixture of polynucleotide fragments, some of which may only have partial oligonucleotide tags because of early termination of the polymerase extension reaction, e.g. by incorporation of a dideoxynucleotide. After such generation, the polynucleotide fragments are separated and fractions are collected. Preferably, only fragments containing complete oligonucleotide tags are processed further and fragments with partial tags are discarded.

### Formation of Tag-Polynucleotide Conjugates and Sampling

An important feature of the invention is the use of oligonucleotide tags consisting of oligonucleotides selected from a minimally cross-hybridizing set of oligonucleotides, or assembled from oligonucleotide subunits selected from a minimally cross-hybridizing set of oligonucleotides. Construction of such minimally cross-hybridizing sets are disclosed in Brenner et al, U.S. Pat. No. 5,846,719, and Brenner et al, Proc. Natl. Acad. Sci., 97: 1665-1670 (2000), which references are incorporated by reference. The sequences of oligonucleotides of a minimally cross-hybridizing set differ from the sequences of every other member of the same set by at least two nucleotides, and more preferably, by at least three nucleotides. Thus, each member of such a set cannot form a duplex (or triplex) with the complement of any other member with less than two mismatches, or three mismatches as the case may be. Preferably, perfectly matched duplexes of tags and tag complements of the same minimally cross-hybridizing set have approximately the same stability, especially as measured by melting temperature. Complements of oligonucleotide tags, referred to herein as “tag complements,” may comprise natural nucleotides or non-natural nucleotide analogs. In one aspect, non-natural nucleic acid analogs are used as tag complements that remain stable under repeated washings and hybridizations of oligonucleoitde tags. In particular, tag complements may comprise peptide nucleic acids (PNAs). Oligonucleotide tags from the same minimally cross-hybridizing set when used with their corresponding tag complements provide a means of enhancing specificity of hybridization.

Minimally cross-hybridizing sets of oligonucleotide tags and tag complements may be synthesized either combinatorially or individually depending on the size of the set desired and the degree to which cross-hybridization is sought to be minimized (or stated another way, the degree to which specificity is sought to be enhanced). For example, a minimally cross-hybridizing set may consist of a set of individually synthesized 10-mer sequences that differ from each other by at least 4 nucleotides, such set having a maximum size of 332, when constructed as disclosed in Brenner et al, International patent application PCT/US96/09513. Alternatively, a minimally cross-hybridizing set of oligonucleotide tags may also be assembled combinatorially from subunits which themselves are selected from a minimally cross-hybridizing set. For example, a set of minimally cross-hybridizing 12-mers differing from one another by at least three nucleotides may be synthesized by assembling 3 subunits selected from a set of minimally cross-hybridizing 4-mers that each differ from one another by three nucleotides. Such an embodiment gives a maximally sized set of 9³, or 729, 12-mers.

When synthesized combinatorially, an oligonucleotide tag preferably consists of a plurality of subunits, each subunit preferably consisting of an oligonucleotide of 3 to 9 nucleotides in length wherein each subunit is selected from the same minimally cross-hybridizing set. In such embodiments, the number of oligonucleotide tags available depends on the number of subunits per tag and on the length of the subunits.

Preferably, tag complements are synthesized on the surface of a solid phase support, such as a microscopic bead or a specific location on an array of synthesis locations on a single support, such that populations of identical, or substantially identical, sequences are produced in specific regions. That is, the surface of each support, in the case of a bead, or of each region, in the case of an array, is derivatized by copies of only one type of tag complement having a particular sequence. The population of such beads or regions contains a repertoire of tag complements each with distinct sequences. As used herein in reference to oligonucleotide tags and tag complements, the term “repertoire” means the total number of different oligonucleotide tags or tag complements. A repertoire may consist of a set of minimally cross-hybridizing set of oligonucleotides that are individually synthesized, or it may consist of a concatenation of oligonucleotides each selected from the same set of minimally cross-hybridizing oligonucleotides. In the latter case, the repertoire is preferably synthesized combinatorially.

When tag complements are attached to or synthesized on microbeads, a wide variety of solid phase materials may be used with the invention, including microbeads made of controlled pore glass (CPG), highly cross-linked polystyrene, acrylic copolymers, cellulose, nylon, dextran, latex, polyacrolein, and the like, disclosed in the following exemplary references: Meth. Enzymol., Section A, pages 11-147, vol. 44 (Academic Press, New York, 1976); U.S. Pat. Nos. 4,678,814; 4,413,070; and 4,046;720; and Pon, Chapter 19, in Agrawal, editor, Methods in Molecular Biology, Vol. 20, (Humana Press, Totowa, N.J., 1993). Microbead supports further include commercially available nucleoside-derivatized CPG and polystyrene beads (e.g. available from Applied Biosystems, Foster City, Calif.); derivatized magnetic beads; polystyrene grafted with polyethylene glycol (e.g., TentaGel™, Rapp Polymere, Tubingen Germany); and the like. Generally, the size and shape of a microbead is not critical; however, microbeads in the size range of a few, e.g. 1-2, to several hundred, e.g. 200-1000 μn diameter are preferable, as they facilitate the construction and manipulation of large repertoires of oligonucleotide tags with minimal reagent and sample usage and also provide enough tag complements to facilitate detection of labeled oligonucleotide tags using conventional detection methods. In one aspect, glycidal methacrylate (GMA) beads available from Bangs Laboratories (Carmel, Ind.) are used as microbeads in the invention. Such microbeads are useful in a variety of sizes and are available with a variety of linkage groups for synthesizing tags and/or tag complements.

As mentioned above, in one aspect tag complements comprise PNAs, which may be synthesized using methods disclosed in the art, such as Nielsen and Egholm (eds.), Peptide Nucleic Acids: Protocols and Applications (Horizon Scientific Press, Wymondham, UK, 1999); Matysiak et al, Biotechniques, 31: 896-904 (2001); Awasthi et al, Comb. Chem. High Throughput Screen., 5: 253-259 (2002); Nielsen et al, U.S. Pat. No. 5,773,571; Nielsen et al, U.S. Pat. No. 5,766,855; Nielsen et al, U.S. Pat. No. 5,736,336; Nielsen et al, U.S. Pat. No. 5,714,331; Nielsen et al, U.S. Pat. No. 5,539,082; and the like, which references are incorporated herein by reference.

Sets containing several hundred to several thousands, or even several tens of thousands, of oligonucleotides may be synthesized directly by a variety of parallel synthesis approaches, e.g. as disclosed in Frank et al, U.S. Pat. No. 4,689,405; Frank et al, Nucleic Acids Research, 11: 4365-4377 (1983); Matson et al, Anal. Biochem., 224: 110-116 (1995); Fodor et al, International application PCT/US93/04145; Pease et al, Proc. Natl. Acad. Sci., 91: 5022-5026 (1994); Southern et al, J. Biotechnology, 35: 217-227 (1994), Brennan, International application PCT/US94/05896; Lashkari et al, Proc. Natl. Acad. Sci., 92: 7912-7915 (1995); or the like.

Preferably, tag complements in mixtures, whether synthesized combinatorially or individually, are selected to have similar duplex or triplex stabilities to one another so that perfectly matched hybrids have similar or substantially identical melting temperatures. This permits mis-matched tag complements to be more readily distinguished from perfectly matched tag complements in the hybridization steps, e.g. by washing under stringent conditions. For combinatorially synthesized tag complements, minimally cross-hybridizing sets may be constructed from subunits that make approximately equivalent contributions to duplex stability as every other subunit in the set. Guidance for carrying out such selections is provided by published techniques for selecting optimal PCR primers and calculating duplex stabilities, e.g. Rychlik et al, Nucleic Acids Research, 17: 8543-8551 (1989) and 18: 6409-6412 (1990); Breslauer et al, Proc. Natl. Acad. Sci., 83: 3746-3750 (1986); Wetmur, Crit. Rev. Biochem. Mol. Biol., 26: 227-259 (1991); and the like. A minimally cross-hybridizing set of oligonucleotides can be screened by additional criteria, such as GC-content, distribution of mismatches, theoretical melting temperature, and the like, to form a subset which is also a minimally cross-hybridizing set.

The oligonucleotide tags of the invention and their complements are conveniently synthesized on an automated DNA synthesizer, e.g. an Applied Biosystems, Inc. (Foster City, Calif.) model 392 or 394 DNA/RNA Synthesizer, using standard chemistries, such as phosphoramidite chemistry, e.g. disclosed in the following references: Beaucage and Iyer, Tetrahedron, 48: 2223-2311 (1992); Molko et al, U.S. patent 4,980,460; Koster et al, U.S. Pat. No. 4,725,677; Caruthers et al, U.S. Pat. Nos. 4,415,732; 4,458,066; and 4,973,679; and the like. Preferably, oligonucleotide tags of the invention are assembled enzymatically as disclosed by Brenner et al, International patent application PCT/US00/20639.

Tag-polynucleotide conjugates are conveniently formed by inserting the set of polynucleotides being analyzed into a vector containing a library of oligonucleotide tags, as shown below (SEQ ID NO: 1).

Formula I

```
          Left Primer Bsp 120I
5′-AGAATTCGGGCCTTAATTAA ↓
5′- AGAATTCGGGCCTTAATTAA- [⁶(A,C,G,T)₄]-GGGCCC-
        TCTTAAGCCCGGAATTAATT- [⁶(T,G,C,A)₄]-CCCGGG-
              ↑ ↑
          Eco RI Pac I
                                                              Bbs I Bam HI
                                                                ↓ ↓
                                                -GCATAAGTCTTCXXX ... XXXGGATCCGAGTGAT -3′
                                                -CGTATTCAGAAGXXX ... XXXCCTAGGCTCACTA
                                                                                            XXXXXCCTAGGCTCACTA-5′
                                                                                                  Right Primer
```

The flanking regions of the oligonucleotide tag may be engineered to contain restriction sites, as exemplified above, for convenient insertion into and excision from cloning vectors. Optionally, the right or left primers may be synthesized with a biotin attached (using conventional reagents, e.g. available from Clontech Laboratories, Palo Alto, Calif.) to facilitate purification after amplification and/or cleavage. Preferably, for making tag-fragment conjugates, the above library is inserted into a conventional cloning vector, such a pUC19, or the like. Optionally, the vector containing the tag library may contain a “stuffer” region, “XXX . . . XXX,” which facilitates isolation of fragments fully digested with, for example, Bam HI and Bbs I.

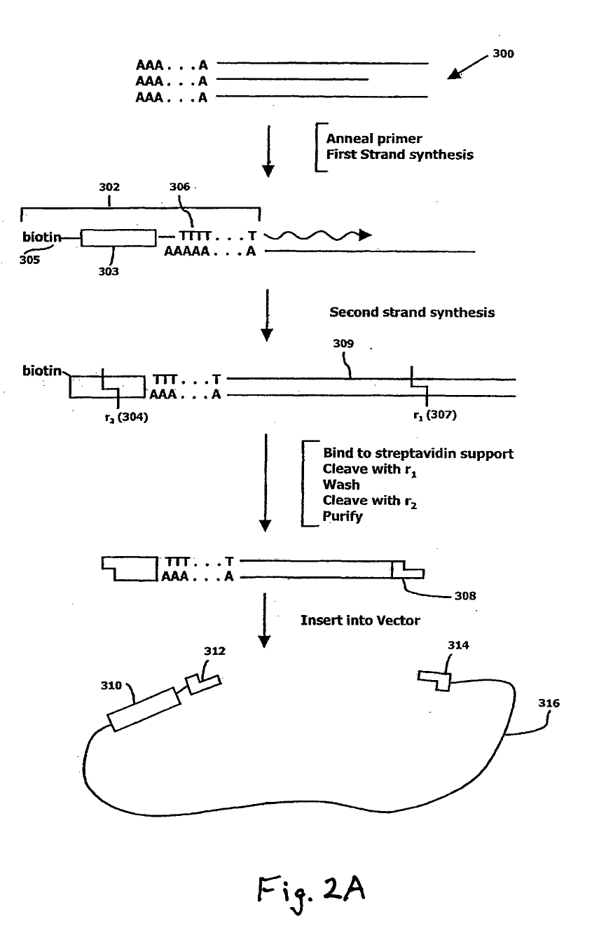

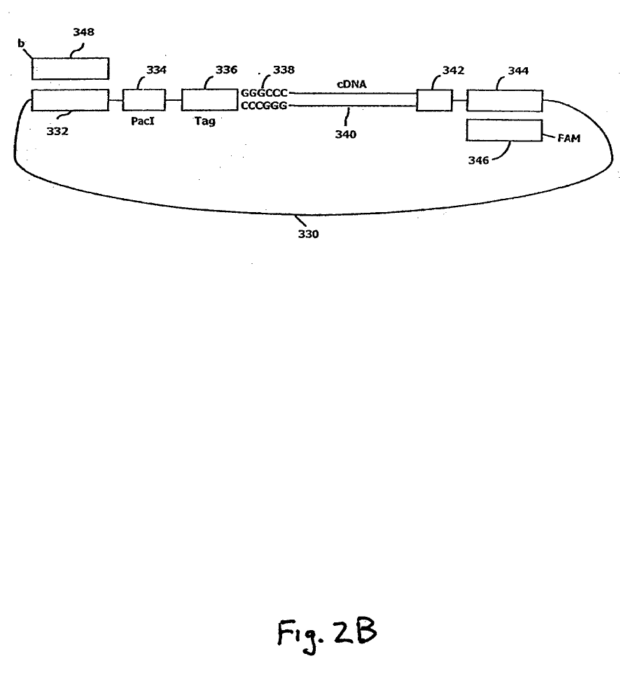

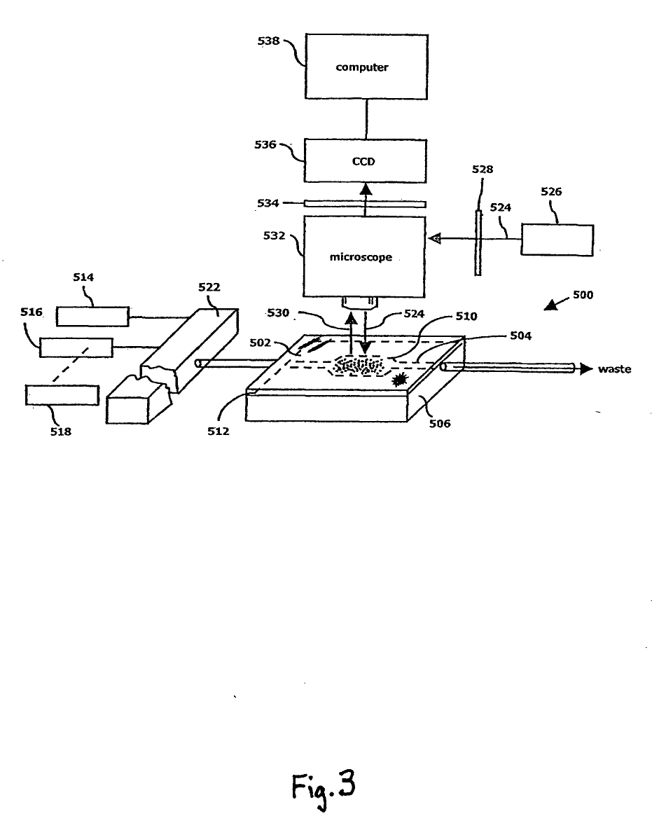

The steps of inserting cDNAs into such a vector are illustrated in FIGS. 2A and 2B. First, mRNA (300) is extracted from a cell or tissue source of interest using conventional techniques and is converted into cDNA (309) with ends appropriate for inserting into vector (316). Preferably, primer (302) having a 5′ biotin (305) and poly(dT) region (306) is annealed to mRNA strands (300) so that the first strand of cDNA (309) is synthesized with a reverse transcriptase in the presence of the four deoxyribonucleoside triphosphates. Preferably, 5-methyldeoxycytidine triphosphate is used in place of deoxycytosine triphosphate in the first strand synthesis, so that cDNA (309) is hemi-methylated, except for the region corresponding to primer (302). This allows primer (302) to contain a non-methylated restriction site for releasing the cDNA from a support. The use of biotin in primer (302) is not critical to the invention and other molecular capture techniques, or moieties, can be used, e.g. triplex capture, or the like. Region (303) of primer (302) preferably contains a sequence of nucleotides that results in the formation of restriction site r₂ (304) upon synthesis of the second strand of cDNA (309). After isolation by binding the biotinylated cDNAs to streptavidin supports, e.g. Dynabeads M-280 (Dynal, Oslo, Norway), or the like, cDNA (309) is preferably cleaved with a restriction endonuclease which is insensitive to hemimethylation (of the C's) and which recognizes site r₁ (307). Preferably, r₁ is a four-base recognition site, e.g. corresponding to Dpn II, or like enzyme, which ensures that substantially all of the cDNAs are cleaved and that the same defined end is produced in all of the cDNAs. After washing, the cDNAs are then cleaved with a restriction endonuclease recognizing r₂, releasing fragment (308) which is purified using standard techniques, e.g. ethanol precipitation, polyacrylamide gel electrophoresis, or the like. After resuspending in an appropriate buffer, fragment (308) is directionally ligated into vector (316), which carries tag (310) and a cloning site with ends (312) and (314). Tag (310) includes a hybridization tag, a primer binding site, and a correlation tag. Preferably, vector (316) is prepared with a “stuffer” fragment in the cloning site to aid in the isolation of a fully cleaved vector for cloning.

After formation of a library of tag-cDNA conjugates, a sample of host cells is usually plated to determine the number of recombinants per unit volume of culture medium. The size of sample taken for further processing preferably depends on the size of tag repertoire used in the library construction, as discussed above. Preferably, tag-cDNA conjugates are carried in vector (330) which comprises the following sequence of elements: first primer binding site (332), restriction site r₃ (334), oligonucleotide tag (336), junction (338), cDNA (340), restriction site r₄ (342), and second primer binding site (344). After a sample is taken of the vectors containing tag-cDNA conjugates the following steps are implemented: The tag-cDNA conjugates may be amplified from vector (330) by use of biotinylated primer (348) and labeled primer (346) in a conventional polymerase chain reaction (PCR) in the presence of 5-methyldeoxycytidine triphosphate, after which the resulting amplicon is isolated by streptavidin capture. Restriction site r₃ preferably corresponds to a rare-cutting restriction endonuclease, such as Pac I, Not I, Fse I, Pme I, Swa I, or the like, which permits the captured amplicon to be release from a support with minimal probability of cleavage occurring at a site internal to the cDNA of the amplicon.

Sampling can be carried out either overtly—for example, by taking a small volume from a larger mixture—after the tags have been attached to the DNA sequences; it can be carried out inherently as a secondary effect of the techniques used to process the DNA sequences and tags; or sampling can be carried out both overtly and as an inherent part of processing steps.

If a sample of n tag-DNA sequence conjugates are randomly drawn from a reaction mixture—as could be effected by taking a sample volume, the probability of drawing conjugates having the same tag is described by the Poisson distribution, P(r)=e^{-λ}(λ)^{r}/r, where r is the number of conjugates having the same tag and λ=np, where p is the probability of a given tag being selected. If n=10⁶ and p=1/(1.67×10⁷) (for example, if eight 4-base words described in Brenner et al were employed as tags), then λ=0.0149 and P(2)=1.13×10⁻⁴. Thus, a sample of one million molecules produces a low expected number of doubles. Such a sample is readily obtained by serial dilutions of a mixture containing tag-fragment conjugates.

Preferably, DNA sequences are conjugated to oligonucleotide tags by inserting the sequences into a conventional cloning vector carrying a tag library. For example, cDNAs may be constructed having a Bsp 120 I site at their 5′ ends and after digestion with Bsp 120 I and another enzyme such as Sau 3A or Dpn II may be directionally inserted into a pUC19 carrying the tags of Formula I to form a tag-cDNA library, which includes every possible tag-cDNA pairing. A sample is taken from this library for analysis. Sampling may be accomplished by serial dilutions of the library, or by simply picking plasmid-containing bacterial hosts from colonies. After amplification, the tag-cDNA conjugates may be excised from the plasmid. The sample of conjugates is used to generate a size ladder of polynucleotide fragments.

Selection of a tag repertoire to be used with the invention is a matter of design choice which may be influenced by several factors, including the number of signature sequences to be determined per operation, i.e. the throughput, the duration of hybridization reaction(s), tolerance to non-specific hybridizations, the number of polynucleotides being analyzed per operation, the size of tag desired, the size of hybridization array available, tolerance to “doubles,” composition of words, and the like. Preferably, a repertoire of tags is selected that is produced by combinatorial synthesis of words. This permits the efficient synthesis of a large number of tags with similar properties. Preferably, a repertoire of tags consists of between about 5×10⁴ and about 2×10⁶ tags of different nucleotide sequences. In other words, the size of the repertoire is preferably between about 5×10⁴ and about 5×10⁶. For samples of tag-polynucleotide conjugates in the range of between about one and about ten percent of the repertoire size, this results in hybridization reactions of mixtures having complexities in the range of from 50 to 5×10⁵ species. That is, such parameter selections require hybridization reactions that involve the formation of a number of detectable duplexes between about 500 and about 5×10⁵. Preferably, as used here, “detectable duplex” means that the signal-to-noise ratio of a signal collected from a labeled tag at a hybridization site is at least 2; more preferably, it is at least 3.

The specificity of the hybridization reactions of tags and tag complements may be increased by selecting words that have a larger number of mismatches between non-perfectly matched sequences. Preferably, tags of the present invention are constructed from 6-mer words selected from the set listed in Table I. Each word of this set forms a duplex with at least four mismatches with the complements of any other word of the same set. In further preference, tags used in the invention are constructed from a concatenation of four words selected from the set of Table I. Preferably, each word is separated from its neighboring word by a “spacer” nucleotide so that the preferred words have the form:

. . . wwwwwwXwwwwwwXwwwwwwXwwwwww . . .

where “w” designates a nucleotide of a word and “X” designates a “spacer” nucleotide. Tags with such a structure give rise to a repertoire size of 32⁴, or 1,048,576 tags. The sequences and melting temperatures of the tags generated by such words are readily listed using computer programs such as that disclosed in Appendix 1. For the set of words of Table I, distributions of melting temperatures were calculated for tags forming perfectly matched duplexes, tags forming duplexes with a mismatch in the 3′-most word, and tags forming duplexes with a mismatch in the 5′-most word (i.e. the most stable of the single word mismatches). The results are shown in Appendix 2, and demonstrate that with such a set of tags, wash temperatures can be selected that above which perfectly matched tag duplexes are stable and below which all tag duplexes containing mismatches are unstable and will dissociate. Preferably, oligonucleotide tag repertoires are constructed as disclosed by Brenner and Williams, International patent publication WO 00/20639, which is incorporated herein by reference.

TABLE I
Minimally cross-hybridizing set of 6-mers that
may be used to form 27-mer tags having one
nucleotide spacers between words

| | | | |
|---|---|---|---|
| ACACTG | CACTGA | GGATTA | TAGCTA |
| ACTGAC | CAGACT | GGTAAT | TATAGC |
| AGGATC | CCATAT | GTAGAG | TCAGGA |
| ATATGC | CCTATA | GTCTCT | TCCTTC |
| ATCGTA | CTCAAC | GTGAGT | TCGAAG |
| ATGCAT | CTGTTC | GTTCTC | TCTCCT |
| ATTACG | GAGTAC | TAATCG | TGCACA |
| CAAGTC | CATGCA | TACGAT | TGGTGT |

TABLE II.
The expected number of signature sequence for different size of tag sets
and for different sizes of samples for analysis. The right-most column
shows the expected number of signature sequences for 50% efficiency in
processing steps.

| | Sample | | Number | Sample Size | |
|---|---|---|---|---|---|
| | Size | Percent | of | Less | With 50% |
| Size of Tag Set | (Approx.) | Doubles | Doubles | Doubles | Efficiency |
| 1,048,526 | | | | | |
| (=32⁴) | | | | | |
| 10% | 105,000 | 0.5 | 525 | 104,000 | 52,000 |
| 20% | 210,000 | 1.7 | 3,570 | 206,000 | 103,000 |
| 30% | 315,000 | 3.7 | 11,655 | 303,000 | 151,000 |
| 40% | 420,000 | 6.2 | 26,040 | 394,000 | 197,000 |
| 50% | 525,000 | 9.0 | 47,250 | 478,000 | 239,000 |
| 810,000 | | | | | |
| (=30⁴) | | | | | |
| 10% | 81,000 | 0.5 | 405 | 80,000 | 40,000 |
| 20% | 162,000 | 1.7 | 2,754 | 159,000 | 79,000 |
| 30% | 243,000 | 3.7 | 8,991 | 234,000 | 117,000 |
| 40% | 324,000 | 6.2 | 20,088 | 304,000 | 152,000 |
| 50% | 405,000 | 9.0 | 36,450 | 368,000 | 184,000 |
| 614,656 | | | | | |
| (=28⁴) | | | | | |
| 10% | 61,000 | 0.5 | 305 | 60,000 | 30,000 |
| 20% | 122,000 | 1.7 | 2,074 | 120,000 | 60,000 |
| 30% | 183,000 | 3.7 | 6,771 | 176,000 | 88,000 |
| 40% | 244,000 | 6.2 | 15,128 | 229,000 | 115,000 |
| 50% | 307,000 | 9.0 | 27,630 | 280,000 | 140,000 |

Hybridization tags of oligonucleotide tags generated in accordance with the invention can be labeled in a variety of ways, including the direct or indirect attachment of fluorescent moieties, colorimetric moieties, chemiluminescent moieties, and the like. Many comprehensive reviews of methodologies for labeling DNA provide guidance applicable to generating labeled oligonucleotide tags of the present invention. Such reviews include Haugland, Handbook of Fluorescent Probes and Research Chemicals, Sixth Edition (Molecular Probes, Inc., Eugene, 2001); Keller and Manak, DNA Probes, 2nd Edition (Stockton Press, New York, 1993); Eckstein, editor, Oligonucleotides and Analogues: A Practical Approach (IRL Press, Oxford, 1991); Wetmur, Critical Reviews in Biochemistry and Molecular Biology, 26: 227-259 (1991); and the like. Particular methodologies applicable to the invention are disclosed in the following sample of references: Fung et al, U.S. Pat. No. 4,757,141; Hobbs, Jr., et al U.S. Pat. No. 5,151,507; Cruickshank, U.S. Pat. No. 5,091,519.

Selection of fluorescent dyes and means for attaching or incorporating them into DNA strands is well known, e.g. Matthews et al, Anal. Biochem., Vol 169, pgs. 1-25 (1988); Haugland, Handbook of Fluorescent Probes and Research Chemicals (Molecular Probes, Inc., Eugene, 2001); Keller and Manak, DNA Probes, 2nd Edition (Stockton Press, New York, 1993); and Eckstein, editor, Oligonucleotides and Analogues: A Practical Approach (IRL Press, Oxford, 1991); Wetmur, Critical Reviews in Biochemistry and Molecular Biology, 26: 227-259 (1991); Ju et al, Proc. Natl. Acad. Sci., 92: 43474351 (1995) and Ju et al, Nature Medicine, 2: 246-249 (1996); and the like.

Preferably, one or more fluorescent dyes are used as labels for the oligonucleotide tags, e.g. as disclosed by Menchen et al, U.S. Pat. No. 5,188,934 (4,7-dichlorofluorscein dyes); Begot et al, U.S. Pat. No. 5,366,860 (spectrally resolvable rhodamine dyes); Lee et al, U.S. Pat. No. 5, 847,162 (4,7-dichlororhodamine dyes); Khanna et al, U.S. Pat. No. 4,318,846 (ether-substituted fluorescein dyes); Lee et al, U.S. Pat. No. 5,800,996 (energy transfer dyes); Lee et al, U.S. Pat. No. 5,066,580 (xanthene dyes): Mathies et al, U.S. Pat. No. 5,688,648 (energy transfer dyes); and the like. As used herein, the term “fluorescent signal generating moiety” means a signaling means which conveys information through the fluorescent absorption and/or emission properties of one or more molecules. Such fluorescent properties include fluorescence intensity, fluorescence life time, emission spectrum characteristics, energy transfer, and the like.

### Hybridization Arrays

Hybridization tags of the invention are detected by specifically hybridizing them to an array of spatially discrete hybridization sites containing complementary sequences. Preferably such arrays are random microarrays, so that the quantities of reactants, e.g. labeled tags, or the like, and the volumes of reagents in the hybridization reaction may be minimized. Such arrays include arrays of microbeads as disclosed by Brenner et al, International patent application PCT/US98/11224. As mentioned above, preferably hybridization arrays of the invention comprise oligonucleotides that are made from nucleotide analogs that permit a large number of cycles of hybridizing and washing of labeled oligonucleotide tags without significant degradation, or loss of signal with successive cycles. Preferably, a hybridization array of the invention can sustain at least 30 cycles of hybridization and washing; and more preferably, at least 50 cycles; and still more preferably, at least 80 cycles. As mentioned above, in one aspect, hybridization arrays of the invention comprise PNA tag complements.

Guidance for selecting conditions and materials for applying labeled oligonucleotide probes to microarrays may be found in the literature, e.g. Wetmur, Crit. Rev. Biochem. Mol. Biol., 26: 227-259 (1991); DeRisi et al, Science, 278: 680-686 (1997); Chee et al, Science, 274: 610-614 (1996); Duggan et al, Nature Genetics, 21: 10-14 (1999); Schena, Editor, Microarrays: A Practical Approach (IRL Press, Washington, 2000); and like references.

Instruments for measuring optical signals, especially fluorescent signals, from labeled tags hybridized to targets on a microarray are described in the following references which are incorporated by reference: Stern et al, PCT publication WO 95/22058; Resnick et al, U.S. Pat. No. 4,125,828; Karnaukhov et al, U.S. Pat. No. ,354,114; Trulson et al, U.S. Pat. No. 5,578,832; Pallas et al, PCT publication WO 98/53300; and the like. An exemplary instrument for carrying out hybridization reactions on microbead arrays is shown in FIG. 5, and is disclosed in detail in Pallas et al (cited above) and Brenner et al, Nature Biotechnology, 18: 630-634 (2000).

### Generation of Size Ladders

In one aspect, target polynucleotides are prepared for signature sequencing as illustrated in FIG. 2A. A conventional library is formed from genomic or other DNA (206) by inserting such DNA (208) into cloning vector (210). Separately, tag vector library (200) is prepared as described above. Each vector of the library contains a hybridization tag (202), a correlation tag (204), and a primer binding site (216) between the two tags as shown (214). Preferably, primer binding site (216) is designed to contain a unique type IIs restriction site for cleaving the vector downstream of the correlation tag to permit insertion of target DNA (208). The two libraries are processed (212) as follows: Target DNA (208) is excised from vector (210), purified, and inserted into a linearized tag vector to produce library containing a conjugate of every tag and every target DNA. A sample of vectors is taken from this conjugate library and amplified, either by cloning or by PCR, to form a library (214) of target DNAs for sequencing. The size of the sample is a design choice for one of ordinary skill in the art that depends on several factors, including the size of the tag library, the number of hybridization sites in the random microarrays employed, the degree of certainty desired for capturing every different target DNA in the sample, the number of doubles that are desired, and the like. Exemplary, sample sizes are listed for three different library sizes in Table II. Preferably, the size of the library is about 10⁶ and a sample of 10⁶ conjugate is taken; thus, about 40% of the tags will be attached to more than one target DNA and will generate more than one signal, and 60% of the hybridization sites will generate a single signal. Hybridization sites corresponding to doubles are ignored, or may be used if optical means, e.g. filters, and the like, are provided for discriminating the multiple signals.

### Separation of Size Ladders by Denaturing HPLC

The following describes a procedure for size-based and sequence-independent separation of extension products from approximately 50 to 100 nucleotides in length.

Preferably, separation is performed by integrated high performance liquid chromatography (HPLC) with a detector-coupled fraction collector and with column and mobile phase gradients optimized for the separation of DNA components into microwell plates. As necessary, separation may employ either diethyl amino ethane (DEAE) anion exchange chromatography, or ion-pairing reverse phase chromatography, or a combination of both to effect the purification. The separation is performed on samples containing as little as 1 nanogram (ng) of each base-size group of oligonucleotides, and containing as much as 1 μg total oligonucleotides, and on samples containing as many as 50 sizes of oligonucleotides to be separated.

The procedure utilizes the following equipment and reagents:

1. High Pressure Liquid Chromatograph—HP1100 (Agilent Technologies) or equivalent, with a minimal configuration consisting of a binary pump, UV detector, Column Heater, and Injection System

2. 96-well based Fraction Collection System, with automated peak detection based control of fraction collection. Manual fraction collection may be substituted.

3. DEAE Ion Exchange Chromatography:

Column—Dionex DNA-PAC (or equivalent)

HPLC Solvents

A) Distilled, deionized water (dH20)

B) Sodium perchlorate (0.375M in dH20)

C) Sodium chloride (2M in dH20)

Typical Conditions—Solvent Flow at 1.0 mL/min., Detector at 260 nm, Column oven at 50° C. Initial solvent conditions are 0% Solvent B and 100% of Solvent A. Upon injection of sample, solvent programmed linearly to 80% B in 60 minutes. Solvent C may be used to optimize separations. Conditions are optimized to provide maximal separation by oligonucleotide size, while minimizing sequence-based separation.

4. Ion Pairing Reverse Phase Chromatography:

Column—Zorbax Eclipse-DNA column (Agilent Technologies), or equivalent

Ion Paring Reagent—Tetraalkyl ammonium bromide, where the alkyl group is typically tetra butyl, however tetra hexyl-, or tetra octyl- may be substituted to obtain optimal separation for a particular library.

HPLC Solvents

A) Distilled, deionized water (dH2O) with typically O.1M ion pairing agent (adjusted for optimal separation for a particular library)

B) Acetonitrile (ACN) with typically 0.1M ion pairing agent (adjusted as above)

Typical Conditions—Solvent Flow at 1.0 mL/min., Detector at 260 nm, Column oven at 50° C. Initial solvent conditions are 20% Solvent B and 80% of Solvent A. Upon injection of sample, solvent programmed linearly to 80% B in 60 minutes. Conditions are optimized to provide maximal separation by oligonucleotide size, while minimizing sequence-based separation.

Procedure:

Samples are concentrated to approximately 0.10 to 1.00 μg total DNA in 20 μL. The HPLC is typically setup using the ion-pairing reverse phase chromatographic conditions above. The 20 μL sample is injected upon the HPLC and the detector output (at 260 nm) is tracked either manually or via computer to direct samples eluting from the column either to waste (before the samples start to elute) or to the microplate fraction collector. At start of elution of DNA peaks, samples are collected, at minimum, one fraction per peak as observed on the HPLC detector output. After elution of constituent DNA peaks, the HPLC column elute is diverted to waste, and the column is washed with 80% of Solvent B.

Alternately, as necessary, a similar procedure is employed with DEAE anion exchange HPLC to pre-separate DNA by size, before transfer of individual eluting peaks to ion pairing reverse phase HPLC for final separation and collection as described above. The procedure may be performed manually or by computer controlled column switching to automate the 2-dimensional size-based purification of DNA libraries.

After collection, DNA size-separated fractions, are purified and concentrated for use in sequencing.

### Instrumentation for Hybridizing Labeled Tags to an Array of Microbeads

Several instruments are available for implementing the method of the invention. In particular, instruments used for hybridizing fluorescent probes to microarrays may be used with the present invention, such as disclosed in U.S. Pat. No. 5,992,591, or like instrument.

When an array of microbeads is used as solid phase supports, apparatus as described in Interntional application PCT/US98/11224 or Brenner et al, Nature Biotechnology, 18: 630-634 (2000), may be used. A flow chamber (500), diagrammatically represented in FIG. 5, is prepared by etching a cavity having a fluid inlet (502) and outlet (504) in a glass plate (506) using standard micromachining techniques, e.g. Ekstrom et al, International patent application PCT/SE91/00327; Brown, U.S. Pat. No. 4,911,782; Harrison et al, Anal. Chem. 64: 1926-1932 (1992); and the like. The dimension of flow chamber (500) are such that loaded microbeads (508), e.g. GMA beads, may be disposed in cavity (510) in a closely packed planar monolayer of 500 thousand to 1 million beads. Cavity (510) is made into a closed chamber with inlet and outlet by anodic bonding of a glass cover slip (512) onto the etched glass plate (506), e.g. Pomerantz, U.S. Pat. No. 3,397,279. Reagents are metered into the flow chamber from syringe pumps (514 through 520) through valve block (522) controlled by a microprocessor as is commonly used on automated DNA and peptide synthesizers, e.g. Bridgham et al, U.S. Pat. No. 4,668,479; Hood et al, U.S. Pat. No. 4,252,769; Barstow et al, U.S. Pat. No. 5,203,368; Hunkapiller, U.S. Pat. No. 4,703,913; or the like.

Hybridization, identification, and washing are carried out in flow chamber (500) to generate signature sequences. Labeled oligonucleotide tags specifically hybridize to tag complements and are detected by exciting their fluorescent labels with illumination beam (524) from light source (526), which may be a laser, mercury arc lamp, or the like. Illumination beam (524) passes through filter (528) and excites the fluorescent labels on tags specifically hybridized to tag complements in flow chamber (500). Resulting fluorescence (530) is collected by confocal microscope (532), passed through filter (534), and directed to CCD camera (536), which creates an electronic image of the bead array for processing and analysis by workstation (538). Preferably, labeled oligonucleotide tags at 25 nM concentration are passed through the flow chamber at a flow rate of 1-2 μL per minute for 10 minutes at 20° C., after which the fluorescent labels carried by the tag complements are illuminated and fluorescence is collected. The tags are melted from the tag complements by passing NEB #2 restriction buffer with 3 mM MgCl₂ through the flow chamber at a flow rate of 1-2 μL per minute at 55° C. for 10 minutes.

### Use of Massively Parallel Signature Sequencing for Genome-Wide Genotyping and Copy Number Measurement

Unraveling the genetic basis of complex traits remains an unsolved problem of immense medical and economic importance. Association studies, in which multiple alleles of populations of affected and unaffected individuals are compared, provide an approach to this problem; however, such studies require the measurement of 30-50,000 markers per individual in populations of 300-400 affected individuals and an equal number of controls, e.g. Kruglyak et al, Nature Genetics, 27: 234-236 (2001); Lai, Genome Research, 11: 927-929 (2001); Cardon et al, Nature Reviews Genetics, 2: 91-99 (2001).

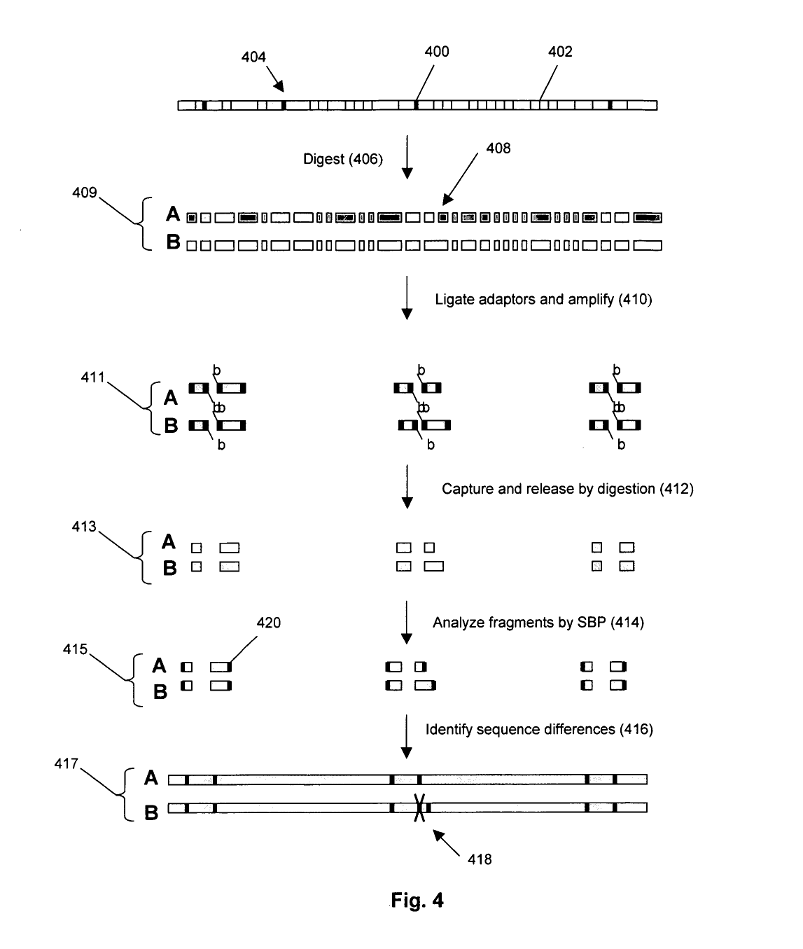

The present invention can make whole genome scans of over a hundred thousand loci in a single operation. Signatures generated by the invention provide sequence tag “addresses” for restriction sites throughout a genome, and such tags can be immediately mapped to loci if a genome sequence is available. Not only can such sequence tags provide SNP information, but they can also measure local amplifications in copy number of specific genomic regions. Whole genome scanning is carried out as follows (as illustrated in FIG. 4), assuming a human genome is being analyzed. First, a subset of genomic fragments, i.e. a partition of a genome, is generated using well-known techniques, e.g. common to amplified restriction fragment polymorphism (AFLP) analysis and representation difference analysis (RDA). In AFLP analysis, a subset is typically created by digesting the genome with an “8-cutter” and “4-cutter” restriction endonucleases. Such a partition of a genome usually comprises an amplicon of a plurality of disjoint fragments, that is, from non-overlapping regions of the genome. This generates about 90,000 fragments having “mixed” ends, that is, an 8-cutter overhang on one end and a 4-cutter overhang on the other end. On average, these fragments are about 256 basepairs in length. Two adaptors are prepared that are ligated to the 8-cutter overhangs and the 4-cutter overhangs, respectively. Each adaptor contains a primer binding site. The primer specific for the 8-cutter adaptor is biotinylated, so that a means is available for separating the amplified fragments having mixed ends from the rest of the reaction mixture. (The number of fragments having two 8-cutter ends is negligible). As in AFLP, the two primers are selected to have 1-2 predetermined nucleotides that extend into the fragment sandwiched between the two adaptors. This is another means for reducing the population of fragments that are amplified. For example, if one primer has a single “T” extension and the other primer has a single “G” extension, then only one sixteenth of the original population of fragments is amplified. (Namely, the fragments having a complementary “C” and a complementary “A” immediately adjacent to 8-cutter and 4-cutter sites at its ends.) In this manner, the original 90,000 mixed-end fragments can be converted into 16 non-overlapping subsets of about 5625 fragments each. After affinity purification with streptavidinated beads, the captured fragments are re-digested with the original 8-cutter and 4-cutter enzymes to release them from the beads. The released fragments are then cloned and tag-fragment conjugates are prepared.

Since sampling the tag-fragment conjugates is a random process, the number of conjugates analyzed must be several fold larger than the size of the fragment set. For example, in order to ensure with >99% probability that all fragments are analyzed, about five times the number of fragments in the set (i.e., 5×5625≈28,000) must be sequenced. Thus, eight of the 5625-fragment populations could be analyzed by SBP in one operation. (Note that a benefit of over-sampling is that on average each signature will be present in five copies, permitting confidence measures to be applied to the data).

The data from SBP provides two types of genotyping information. Genotyping information comes both from the signature sequence itself and from the presence or absence of a restriction site, which is detected by the presence or absence of its associated signature sequence. Thus, each signature actually is a survey of 36(=8+24+4) nucleotides; namely, the 8-cutter site, the 24-nucleotide SBP signature sequence, and the 4-cutter site.

Common SNPs (present at a frequency of >20%) are of particular interest because they can be used in SNP-trait association studies. Common SNPs appear at a rate of about 1 per 1000 basepairs. Since 8.1 MB are surveyed in one SBP run, on average, 8100 common SNPs will be assayed, whether they were known beforehand or not. The “open system” property of SBP provides a significant advantage when there is little knowledge of the identities of common SNPs in a population.

As mentioned above, for larger genomes, such as human genomes, preferably the method of the invention is applied to a representation of the genome in order to reduce the complexity of the reactions. This is conveniently accomplished by amplifying a subset of restriction fragments after digestion with more than one, preferably two, restriction endonucleases. Conveniently, such digestion partitions a genome into several disjoint subsets so that the method of the invention may be applied to each of the subsets of fragments successively to obtain sequence marker frequencies at successively higher densities of loci. Alternatively, different populations of fragments can be generated by using different sets of restriction endonucleases for the digestion. Preferably, for larger genomes restriction endonuclease having a eight-basepair recognition site (“8-cutter”) is used together with a restriction endonuclease having a four-basepair recognition site (“4-cutter”). Exemplary restriction endonucleases having eight-basepair recognition sites include CciNI, FseI, NotI, PacI, SbfI, SdaI, SgfI, Sse8387I, and the like. Exemplary restriction endonucleases having four-basepair recognition sites include Tsp509I, MboI, Sau3AI, DpnII, MaeII, HpaII, MspI, BfaI, HinP1I, TaqI, MseI, HhaI, TaiI, NlaIII, ChaI, and the like. For example, in a genome of about 3×10⁹ basepairs, an 8-cutter will have about 4.6×10⁴ sites, assuming a random occurrence of the different nucleotides throughout the genome. If the genome is digested with both an 8-cutter and a 4-cutter and only fragments having one 8-cutter end and one 4-cutter end are amplified, then about 2×4.6×10⁴ fragments will be amplified for analysis. On average the fragments will be about 128 basepairs in length; thus, about 11.8 MB (=2×128×4.6×10⁴) of sequence will be amplified, or about a 0.4% sample of the genome. Polymorphisms detected by probes directed to these fragments will be uniformly distributed over the genome with an average distance about the same as the distance between the 8-cutter sites, or about 65 kilobases. This average distance can be reduced by using additional 8-cutters. For example, using NotI and Tail and then using Sbfl and Sau3A separately leads to a uniform distribution of sequence markers having an average distance of about 32 kilobases. The selection of combinations of restriction endonucleases to achieve a desired density of sequence markers and complexity of hybridization reactions in a given embodiment is a matter of design choice for one skilled in the art.

FIG. 4 illustrates how signature sequencing of restriction fragments by SBP is used to detect and map restriction site polymorphisms in connection with a genome-wide scan. 8-cutter sites (thick lines, 400) and 4-cutter sites (thin lines, 402) are illustrated in genome segment (404) of a sequenced genome. The availability of a sequenced genome allows SBP sequence tags to be mapped immediately by simply matching signature sequences with segments of the genome sequence in a database. Separately, genomes (404) from populations to be compared are digested (406) as described above to give two populations of fragments (409), A and B. Adaptors are ligated to A & B fragments, then amplified (410) with selective primers, one of which is biotinylated to give populations (411). The biotinylated fragments are captured and the amplified segments of genomic DNA are releasedby digesting the captured population using the same enzymes as used in step (406). Biotinylated fragments are separated by capturing with avidinated beads, after which fragments are released by re-digestion.

What is claimed is:

1. A method of determining nucleotide sequences of a population of polynucleotides, the method comprising the steps of.
attaching an oligonucleotide tag from a repertoire of tags to each polynucleotide of the population to form tag-polynucleotide conjugates;
generating a size ladder of polynucleotide fragments for each tag-polynucleotide conjugate by an extension reaction, each polynucleotide fragment of the same size ladder having an end and the same oligonucleotide tag as every other polynucleotide fragment of the size ladder and each polynucleotide fragment for each tag-polynucleotide conjugate differing in length by one or more nucleotides;
separating the polynucleotide fragments to form a plurality of fractions;
copying and labeling the oligonucleotide tag of each polynucleotide fragment in each fraction according to the identity of one or more nucleotides at the end of such polynucleotide fragments;
hybridizing the labeled oligonucleotide tags of each fraction with their respective complements tinder stringent hybridization conditions, the respective complements each being attached to a spatially discrete region on a solid phase support; and
detecting a sequence of signals from the labels of oligonucleotide tags hybridized to the solid phase support to determine the nucleotide sequences of the polynucleotides of the population.

2. The method of claim 1 wherein said step of separating includes separating each of said polynucleotide fragment of the same size ladder so that it forms a distinct peak relative to other polynucleotide fragments of its size ladder.

3. The method of claim 1 wherein said solid phase support is a microarray.

4. The method of claim 1 wherein said solid phase support is a random microarray.

5. The method of claim 1 wherein said step of labeling includes labeling oligonucleotide tags of polynucleotide fragments having different nucleotides at their ends with labels that generate distinguishable optical signals.

6. The method of claim 5 wherein said step of labeling includes labeling oligonucleotide tags of polynucleotide fragments having the same nucleotides at their ends with labels that generate identical optical signals.

7. The method of claim 5 or 6 wherein said step of detecting includes discarding said sequence of signals from any said spatially discrete region from which more than one said distinguishable optical signals are detected simultaneously.

8. The method of claim 1 wherein said step of separating is carried out by preparative gel electrophoresis or HPLC.

9. The method of claim 8 wherein said step of separating is carried out by denaturing HPLC.

10. A method of determinining nucleotide sequences of a population of polynucleotides, the method comprising the steps of:
generating a size ladder of polynucleotide fragments by an extension reaction, each polynucleotide fragment of the same size ladder having an end and an oligonucleotide tag that is the same for every polynucleotide fragment of the size ladder, the oligonucleotide tag being selected from a minimally cross-hybridizing set of oligonucleotides;
separating the polynucleotide fragments to form a plurality of fractions;
copying and labeling the oligonucleotide tag of each polynucleotide fragment in each fraction according to the identity of one or more nucleotides at the end of such polynucleotide fragments;
hybridizing the labeled oligonucleotide tags of each fraction with their respective complements under stringent hybridization conditions, the respective complements each being attached to a spatially discrete region on a solid phase support; and
detecting a sequence of signals from the labels of oligonucleotide tags hybridized to the solid phase support to determine the nucleotide sequences of the polynucleotides of the population.

11. The method of claim 10 wherein said step of labeling includes labeling oligonucleotide tags of polynucleotide fragments having different nucleotides at their ends with labels that generate distinguishable optical signals.

12. The method of claim 11 wherein said step of detecting includes discarding said sequence of signals from any said spatially discrete region from which more than one said distinguishable optical signals are detected simultaneously.

13. The method of claim 10 wherein said step of hybridizing includes separately hybridizing said labeled oligonucleotide tags of each said fraction with their respective complements under stringent hybridization conditions, recording a signal from each of said hybridized oligonucleotide tags, and washing said solid phase support so that said labeled oligonucleotide tags are removed.

14. A method of monitoring a population of polynucleotides in a reaction using oligonucleotide tags, the method comprising the steps of:
forming tag-polynucleotide conjugates between polynucleotides of the population and oligonucleotide tags of a tag repertoire such that substantially every oligonucleotide tag of the repertoire forms a tag-polynucleotide conjugate with substantially every polynucleotide of the population;
isolating a sample of the tag-polynucleotide conjugates having a size less than or substantially equal to that of the tag repertoire;
conducting a reaction with a plurality of reaction outcomes on the sample, such that each tag-polynucleotide conjugate of the sample has a single reaction outcome;
copying and labeling each oligonucleotide tag of a tag-polynucleotide conjugate according to its reaction outcome such that tag-polynucleotide conjugates having different reaction outcomes have oligonucleotide tags with distinguishable labels;
hybridizing the labeled oligonucleotide tags of each tag-polynucleotide conjugate with their respective complements under stringent hybridization conditions, the respective complements each being attached to a spatially discrete region on a solid phase support; and
detecting signals from the labels of oligonucleotide tags hybridized to the solid phase support to determine reaction outcomes of the polynucleotides of the population.

15. The method of claim 10 wherein said step of labeling includes labeling oligonucleotide tags of polynucleotide fragments having different nucleotides at their ends with labels that generate distinguishable optical signals.

16. A method of measuring relative genomic amplification over a genome, the method comprising the steps of:
providing a partition of a genome, the partition comprising a plurality of fragments uniformly distributed over the genome, each fragment having a genomic location;
generating a signature sequence from each fragment; and
tabulating signature sequences of the fragments at each genomic location; and
determining relative genomic amplification by a relative abundance of each fragment from the tabulated signature sequences.


SEQUENCE LISTING (1 sequence)

SEQ ID NO: 1 - DNA, 89 bp, Artificial Sequence
  primer_bind 71-76: element of cloning vector
agaattcgggccttaattaaddddddddddddddddddddddddddddddddgggcccgcataagtcttcnnnnnnggatccgagtgat